(19) United States
(12) Patent Application Publication
(10) Pub. No.: US 2017/0027861 A1
(43) Pub. Date: Feb. 2, 2017

(54) METHOD OF TREATING T CELL MEDIATED DISORDERS

(71) Applicant: CASE WESTERN RESERVE UNIVERSITY, Cleveland, OH (US)

(72) Inventors: M. Edward Medof, Cleveland, OH (US); Michael G. Strainic, Cleveland, OH (US); Kristina V. Thomas, Cleveland, OH (US); Jodi Arth, Cleveland, OH (US); Fengqi An, Cleveland, OH (US); Juang Huang, Cleveland, OH (US)

(21) Appl. No.: 15/226,652

(22) Filed: Aug. 2, 2016

Related U.S. Application Data
(63) Continuation of application No. 12/921,308, filed on Nov. 23, 2010, now abandoned.
(60) Provisional application No. 61/034,303, filed on Mar. 6, 2008.

(51) Int. Cl. A61K 9/00; A61K 39/395

(52) U.S. Cl. CPC A61K 9/0051; A61K 39/3955

(57) ABSTRACT

A method of treating a T-cell mediated disorders in a tissue includes administering to the tissue of the subject a therapeutically effective amount of a complement antagonist that substantially reduces T-cell differentiation or t-cell inflammatory cytokine generation.

### RELATED APPLICATION

This application claims priority from U.S. Provisional Application No. 61/034,303, filed Mar. 6, 2008, the subject matter, which is incorporated herein by reference in its entirety.

### TECHNICAL FIELD

The present invention relates to a method of treating a T-cell mediated disorder and particularly, to a method of treating a T-cell mediated inflammation.

### BACKGROUND

Herpes stromal keratitis (recurrent infection of the cornea by herpes simplex virus) is the most common cause of infectious conical blindness in the western world. In the US, it is estimated that 400,000 persons are affected, with 20,000 new cases of herpetic stromal keratitis occurring annually. Each episode of herpetic stromal keratitis increases the risk of future episodes of the disease. Current treatment consists of topical steroids in addition to prophylactic oral (acyclovir or valacyclovir) or topical (trifluridine) anti-viral drug therapy. Despite this treatment, patients develop severe corneal scarring due to repeated episodes of the disease, which often require conical transplantation.

Although the disease is initiated by viral infection of corneal cells, the pathology is not caused by the virus itself. It rather is caused by host T cell responses to virally infected corneal cells. Steroids function to inhibit these response but cannot completely block them.

### SUMMARY OF THE INVENTION

The present invention relates to a method of treating T-cell mediated inflammation in a tissue of a subject. The method includes administering to the tissue at least one complement antagonist. The at least one complement antagonist substantially reduces or substantially inhibits at least one of T-cell differentiation or T-cell cytokine expression in the tissue of the subject.

Another aspect of the invention relates to a method of treating T-cell mediated corneal inflammation in a subject by administering to the cornea of the subject at least one complement antagonist. The at least one complement antagonist substantially reduces or substantially inhibits at least one of T-cell differentiation or T-cell cytokine expression in or proximate the cornea of the subject.

A further aspect of the present invention, relates to a method of treating T-cell mediated corneal inflammation in a subject by administering to the cornea of a subject a therapeutically effective amount of at least one complement antagonist that substantially reduces or substantially inhibits the interaction of at least one of C3a or C5a with a C3a receptor (C3aR) and C5a receptor (C5aR) on a T-cell in or proximate the cornea.

Yet another aspect of the invention relates to a method of treating T-cell mediated corneal inflammation associated with Herpes stromal keratits in a subject. The method includes administering to the cornea of the subject at least one complement antagonist directed against at least one of C3, C5, C3 convertase, C5 convertase, C3a, C5a, C3aR, or C5aR. The complement antagonist substantially reduces or substantially inhibits at least one of T-cell differentiation or T-cell cytokine expression in or proximate the cornea.

Another aspect of the invention relates to a method of substantially reducing T-cell inflammatory cytokine expression. The method includes administering to the T-cell at least one complement antagonist that substantially reduces or substantially inhibits interaction of at least one of C3a or C5a with a C3a receptor (C3aR) and C5a receptor (C5aR) of the T-cell.

A further aspect of the invention relates to an ophthalmic preparation for treating T-cell mediated corneal inflammation. The ophthalmic preparation includes an ophthalmic solution and a therapeutically effective amount of at least one complement antagonist directed against at least one of C3, C5, C3 convertase, C5 convertase, C3a, C5a, C3aR, or C5aR that substantially reduces or substantially inhibits at least one of T-cell differentiation or T-cell cytokine expression.

### BRIEF DESCRIPTION OF THE DRAWINGS

FIG. 1 is a schematic illustration of T-cell/antigen presenting cell (APC) partner complement interactions.

FIGS. 2(A-E) illustrate tables, plots, and histograms showing APC-T cell partners upregulate complement mRNAs and the RNAs produce complement proteins. (A) OT-II T cells were incubated for 1 hr with WT DCs±0.1 mM OVA₃₂₃₋₃₃₉ and flow separated (with anti-CD3 and anti-CD11c,) and complement mRNA expression in each partner was measured by qPCR. (B) OT-II cells and DCs were flow separated at increasing times, and complement IL-2,, IFN-g, IL-12, and IL-23 gene expression was measured by qPCR. (C) The left side shows representative (rep) histograms (four exps; linear scales) depicting C5aR or C3aR on OT-II cells and DCs before (no OVA) and after 1 hr interaction with OVA. The right side shows that after 24 hr of interaction of DCs with OT-II cells±OVA, flow-separated cells were cultured for 4 hr, and supernatants were blotted for C3a and C5a; stds=2 ng. (D) Kinetics of C5aR, C3aR, and DAF protein expression on OT-II T cells and DCs during interaction with ova. Fold increase is relative to no OVA cultures. DAF levels on the DCs were low at all time points. (E) After interaction of OT-II cells with DCs±ova for 18 hr with 4 mg/ml anti-B7.1 and anti-B7.2 mAbs or control IgG, mRNAs in flow-separated cells were assayed for C3, C3aR, C5aR, and IFNγ gene expression by qPCR. In parallel cultures, IFNγ was assessed by ELISPOT. No complement or cytokine upregulation occurred without T cells. Data are normalized to no OVA. Each experiment is representative of two to four replicate studies. *p<0.05 versus controls. All error bars are ±SD.

FIGS. 3(A-D) illustrate tables and plots showing disabling C3aR and C5Ar prevents T-cell immunity in vitro. (A) OT-II T cells were incubated for 48 hr with WT DCs and OVA₃₂₃₋₃₃₉±10 ng/ml C5aR-A and 10 ng/ml C3aR-A and either flow separated and assayed for mRNA expression by qPCR (left and middle) or assayed for IFNγ+ cells by ELISPOT (right; dots represent overlapping replicates, n=5 per group). (B) OT-II T cells were incubated for 48 hr at 37° C. with WT, C3ar1^{-/-}, C5ar1^{-/-}, C5ar1^{-/-}, C3ar1^{-/-}, or C3^{-/-}Hc^{-/-} DCs+OVA₃₂₃₋₃₃₉ and assayed for IFNγ⁺ cells by ELISPOT. (C) Purified WT, C3ar1^{-/-}, C5ar1^{-/-}, or C5ar1^{-/-}C3ar1−/− T cells (>96% CD3⁺) were stimulated with 1 mg/ml anti-CD3+1 mg/ml anti-CD28 for 3 hr and assayed for IFNγ+ cells (n=5 each group; some dots overlap. (D) OT-II T cells were incubated for 48 hr at 37° C. with WT or C3ar1^{-/-}C5ar1^{-/-} DCs in the presence of 10 ng/ml C5aR-A and C3aR-A or 10 mg/ml anti-C5a+C3a mAbs, after which IFN-γ producing cells were assayed by ELISPOT. Each experiment was repeated at least twice with comparable results. *p<0.05. All error bars are ±SD.

FIGS. 4(A-F) illustrate tables, plots, and pictures showing the absence of C3aR and C5aR Prevents T Cell Immunity In Vivo (A) WT or C5ar1^{-/-}C3ar1^{-/-} mice (n=3 per group) were immunized with ovalbumin protein mixed in IFA, and spleen cells on day 10 were assayed for responses to OVA₃₂₃₋₃₃₉ by ELISPOT. No response occurred with control peptides or nave mice. (B) Analogous to the experiment in (A), animals were injected s.c. with ovalbumin mixed in PBS, and responses to OVA₃₂₃₋₃₃₉ were assayed on day 10. (C) Syngeneic WT and C5ar1^{-/-}C3ar1^{-/-} male spleen cells were injected i.v. into WT or C5ar1^{-/-}C3ar1^{-/-} B6 females, respectively, and 10 days later, recipient spleen cells were assayed for responses to class II-restricted Dby peptide. (D) WT or C5ar1^{-/-}C3ar1^{-/-} mice were infected with T gondii. All C5ar1^{-/-}C3ar1^{-/-} mice died by day 12, whereas WT animals survived for >50 days. Spleen cells from C5ar1^{-/-}C3ar1^{-/-} and WT animals isolated on day 7 or 10 were stimulated with 1 mg of Toxoplasma gondii antigen and assayed for IFNγ by ELISPOT (left) or for IL-12 by ELISA (right) (n=5 per group each time; some dots overlap). *p<0.05 versus controls. (E) Clinical scores in WT and C5ar1^{-/-}C3ar1^{-/-} mice in which EAE was induced by immunization s.c. with 200 mg MOG35-55 in CFA and 200 ng of pertussis toxin. (n=5 each group, p<0.05). (F) Globes from WT and C5ar1^{-/-}C3ar1^{-/-} mice 15 days after inoculation of scratched corneas with KDS strain of herpes simplex virus (n=5 each group). All error bars are ±SD.

FIGS. 5(A-C) illustrate tables, plots, and histograms showing locally produced C5a and C3a interact with C5aR and C3aR in an autocrine and paracrine fashion to augment T-cell immunity. (A) OT-II T cells were incubated for 1 hr with WT DCs and 0.1 mM OVA₃₂₃₋₃₃₉±10 ng/ml C5aR-A and 10 ng/ml C3aR-A and either flow separated and assayed for mRNA expression (C3, C5aR, C3aR; left and middle) or analyzed (after 24 hr) for C3aR and C5aR by flow cytometry (right); C3aR and C5aR levels on peritoneal macrophages are 14- and 13-fold higher. (B) The left side shows that complement-gene expression was assessed in purified WT T cells after 1 hr stimulation with 1 mg/ml anti-CD3+1 mg/ml anti-CD28±10 ng/ml of C5aR-A and C3aR-A. The right side shows that C3 expression was compared between purified WT and C5ar1^{-/-}C3ar1^{-/-} T cells stimulated with 1 mg/ml each of anti-CD3+anti-CD28. (C) Supernatants from (B) were assayed for C5a+C3a by immunoblotting (std=2 ng).

FIGS. 6(A-C) illustrate table and immunoblots showing C5aR and C3aR Ligation Activates PI3-Kγ, which in turn promotes AKT Phosphorylation. (A) The left side shows that WT T cells were activated with anti-CD3+anti-CD28, and at progressively increasing times, buffer, C5aR-A, or C5aRA+C3aR-A were added; extracts were analyzed for phospho-Ser473 AKT by Luminex assays (representative of two experiments). (p<0.5). The right side shows that naive WT and C5ar1−/−C3ar1−/− T cells were incubated with 1 μg/ml of anti-CD3+anti-CD28 at 37° C. and phospho-Ser473 AKT assessed at increasing times. (B) WT T cells were incubated at 37° C. with anti-CD3+anti-CD28 (1 μg/ml each) for 3 min after which the cells were incubated for 20 min with buffer or 0.1 μM PI-3Kγ-specific inhibitor PI-103. Extracts were immunoblotted with anti-phospho-Ser473 AKT or total AKT mAb (representative of five experiments). (C) OT-II T cells were incubated with 0.1 μM OVA323-339, WT DCs, and 0.1 μM PI-103±10 ng/ml C5a. Complement, IL-2, and IFNγ mRNAs were quantitated by qPCR. All error bars are ±SD.

FIGS. 7(A-F) illustrate tables and plots showing constitutive C5a and C3a production and signaling via the C5aR and C3aR GPCRs influences cell viability in vitro and in vivo. (A) WT T cells were incubated at 37° C. for 17 hr, supernatants were concentrated 10-fold, and C3a or C5a were immunoblotted. The right side shows that WT and C5ar1^{-/-}C3ar1^{-/-} T cells were incubated in complete RPMI 1640 and viability assessed as described in the Experimental Procedures (representative of three experiments). (B) WT and C5ar1^{-/-}, C3ar1^{-/-}, and C5ar1^{-/-}C3ar1^{-/-} T cells were incubated for 6 hr in serum-free HL-1 medium and viability assayed. (C) WT T cells were incubated at 37° C. for 6 hr in HL-1 medium±anti-C3a, anti-C5a, or anti-C3a+anti-C5a and viability assessed. (D) C3^{-/-} and C5-deficient T cells were incubated for 6 hr in HL-1 medium±300 ng/ml C5a added at time 0 and 90 min. (E) Spleens from naive WT, C3ar1^{-/-}, C5ar1^{-/-}, and C3ar1^{-/-}C5ar1^{-/-} mice were isolated (three to five animals per group), total cell numbers were counted, and the CD3⁺ fraction was determined by flow cytometry (WT=10%, C5ar1^{-/-}=5.4%, C3ar1^{-/-}=6.3, and C5ar1^{-/-}C3ar1^{-/-}=4.1%). (F) Immediately after isolation and labeling, 10⁶ CFSE-labeled WT T cells and CellTracker Red CMTPX-labeled C5ar1^{-/-}C3ar1^{-/-} T cells were adoptively cotransferred into the same SCID mice (n=4 mice per group for each time point). Surviving cells numbers were assayed at increasing times. Repeated experiments switching the dyes gave the same results (data not shown). All experiments were performed at least twice with comparable results. *p<0.5 WT versus C5ar1^{-/-}C3ar1^{-/-} cells. All error bars are ±SD.

FIG. 8 illustrates photographs showing the severity of corneal blindness in mice with Herpes Simplex Stromal Keratitis.

FIG. 9 illustrates photographs showing the neovascularization of in mice with Herpes Simplex Stromal Keratitis.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present invention, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, and all “mimetic” and “peptidomimetic” polypeptide forms.

As used herein, the term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

As used herein, the term “antibody” refers to whole antibodies, e.g., of any isotype (IgG, IgA, IgM, IgE, etc.), and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized, and may further include engineered complexes that comprise antibody-derived binding sites, such as diabodies and triabodies.

As used herein, the term “complementary” refers to the capacity for precise pairing between two nucleobases of a polynucleotide and its corresponding target molecule. For example, if a nucleobase at a particular position of a polynucleotide is capable of hydrogen bonding with a nucleobase at a particular position of a target polynucleotide (the target nucleic acid being a DNA or RNA molecule, for example), then the position of hydrogen bonding between the polynucleotide and the target polynucleotide is considered to be complementary. A polynucleotide and a target polynucleotide are complementary to each other when a sufficient number of complementary positions in each molecule are occupied by nucleobases, which can hydrogen bond with each other. Thus, “specifically hybridizable” and “complementary” are terms which can be used to indicate a sufficient degree of precise pairing or complementarity over a sufficient number of nucleobases such that stable and specific binding occurs between a polynucleotide and a target polynucleotide.

As used herein, the term “subject” refers to any warm-blooded organism including, but not limited to, human beings, rats, mice, dogs, goats, sheep, horses, monkeys, apes, rabbits, cattle, etc.

As used herein, the terms “complement polypeptide” or “complement component” refer to a polypeptide (or a polynucleotide encoding the polypeptide) of the complement system that functions in the host defense against infections and in the inflammatory process. Complement polypeptides constitute target substrates for the complement antagonists provided herein.

As used herein, the term “complement antagonist” refers to a polypeptide, polynucleotide, or small molecule capable of substantially reducing or inhibiting the activity of a complement component.

A complement component can include any one or combination of interacting blood polypeptides or glycoproteins. There are at least 30 soluble plasma polypeptides, in addition to cell surface receptors, which can bind complement reaction products and which can occur on inflammatory cells and cells of the immune system. In addition, there are regulatory membrane proteins that can protect host cells from accidental complement attack. Complement components can include polypeptides that function in the classical pathway, such as C2, polypeptides that function in the alternative pathway, such as Factor B, and polypeptides that function in the lectin pathway, such as MASP-1.

Complement components can also include: any of the “cleavage products” (also referred to as “fragments”) that are formed upon activation of the complement cascade; complement polypeptides that are inactive or altered forms of complement polypeptides, such as iC3 and C3a-desArg; and components indirectly associated with the complement cascade. Examples of such complement components can include, but are not limited to, C1q, C1r, C1s, C2, C3, C3a, C3b, C3c, C3dg, C3g, C3d, C3f, iC3, C3a-desArg, C4, C4a, C4b, iC4, C4a-desArg, C5, C5a, C5a-des-Arg, C6, C7, C8, C9, MASP-1, MASP-2, MBL, Factor B, Factor D, Factor H, Factor I, CR1, CR2, CR3, CR4, properdin, C1Inh, C4bp, MCP, DAF, CD59 (MIRL), clusterin, HRF, and allelic and species variants of any complement polypeptide.

As used herein, the terms “treatment,” “treating,” or “treat” refers to any specific method or procedure used for the cure of, inhibition of, prophylaxis of, reduction of, elimination of, or the amelioration of a disease or pathological condition (e.g. corneal inflammation) including, for example, preventing corneal inflammation from developing, inhibiting corneal inflammation development, arresting development of clinical symptoms associated with corneal inflammation, and/or relieving the symptoms associated with corneal inflammation.

As used herein, the term “effective amount” refers to a dosage of a complement antagonist administered alone or in conjunction with any additional therapeutic agents that are effective and/or sufficient to provide treatment of corneal inflammation and/or a disease or disorder associated with corneal inflammation. The effective amount can vary depending on the subject, the disease being treated, and the treatment being affected.

As used herein, the term “therapeutically effective amount” refers to that amount of a complement antagonist administered alone and/or in combination with additional therapeutic agents that results in amelioration of symptoms associated with T-cell mediated inflammation and/or a disease or disorder associated with T-cell mediated corneal inflammation and/or results in therapeutically relevant effect. By way of example, a “therapeutically effective amount” may be understood as an amount of complement antagonist required to reduce corneal inflammation in a subject.

As used herein, the terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrastemal injection and infusion.

As used herein, the terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

The present invention relates generally to immunotherapy, and more particularly to a method of treating T-cell mediated disorders, including but not limited to T-cell mediated ophthalmic or ocular disorders and T-cell mediated corneal inflammation, using complement antagonists. It was found that complement that is locally produced by APCs and T cells during cognate interactions is integrally involved in the T cell activation process. Specifically, C5a and C3a generated from this endogenous production interact with C5aR and C3aR on both APCs and T cells, and these engagements participate in activation and cytokine production by both partners. Previous studies have regarded complement as being separate from T cells and APCs, attributing complement's effects on either cell coming from serum complement rather than from the interacting APCs and T cells themselves, i.e., from the outside-in rather than from the inside of APCs and T cells themselves.

The absence or blockade of C5aR+C3aR leads to lower class II MHC and costimulatory-molecule expression. C5aR and C3aR on T-cells exhibit overlapping but not fully redundant functions because inhibition or deficiency of both has a significantly more profound effect than the absence or blockade of either alone. The foregoing Examples explain why T cell immunity is diminished but not as fully abrogated in the absence of C3, C5, C3aR, or C5aR individually. These findings establish that C5a-C5aR+C3a-C3ar interactions function via both CD28 activation-dependent and -independent mechanisms. CD28 dependence is supported by the observation that CD28 and CD40L engagements upregulate complement and downregulate DAF and that C5a can compensate for CD28 and CD40L blockade or absence. They are consistent with C5a acting both to upregulate costimulatory molecules and to substitute for C5aR+C3aR signaling induced by B7 and CD40 ligation of T cell CD28 and CD40L. APC deficiency of C3, C5aR, and C3aR (each of which leads to less C5a+C3a) markedly limits T cell proliferation and differentiation both in vitro and in vivo. These GPCR interactions additionally function independently of CD28 and is shown by the findings that they are needed to maintain sufficient AKT phosphorylation to support T cell survival: Naive T cells constitutively generate C5a and C3a, C5ar1^{-/-}C3ar1^{-/-} double-deficient cells exhibit reduced viability in vitro and in vivo, addition of C5aR-A+C3aR-A or anti-C5a+anti-C3a mAbs reduces viability of WT T cells, and added C5a promotes viability of Hc^{-/-} and C3^{-/-} T cells.

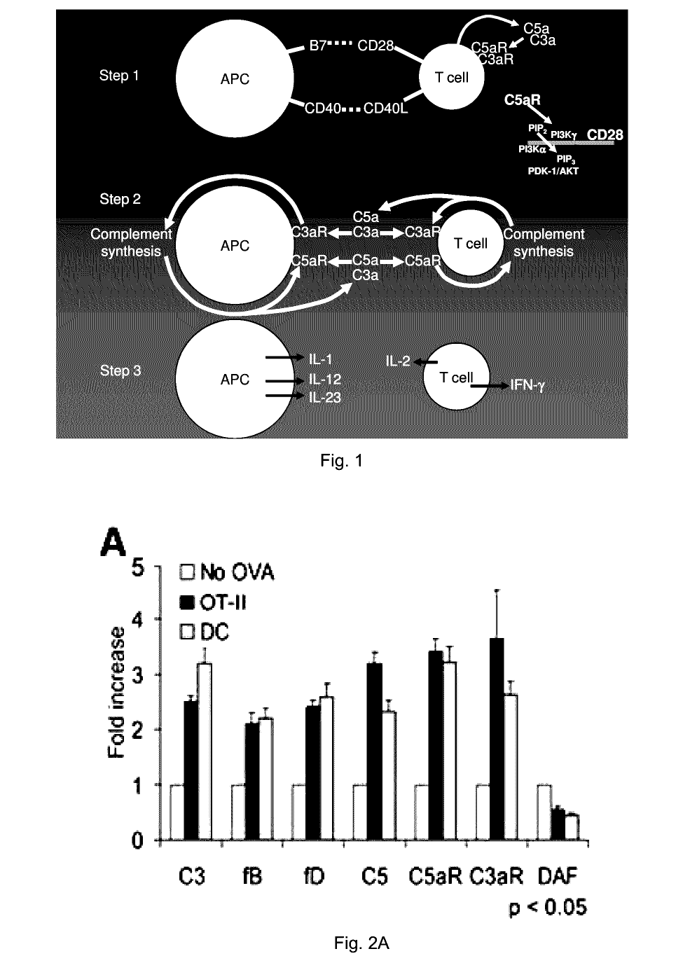

It was also found that deficient C5aR+C3aR expression suppresses IL-2 and IFNγ generation, and disabled C5aR+C3aR GPCR signaling in APCs suppresses IL-1, IL-12p35, and IL-23p19 production. The findings regarding local APC-T cell C5a-C5aR+C3a-C3aR interactions thus identify previously uncovered GPCR steps that participate in both T cell and APC cytokine signaling pathways. Models of initial T cell costimulation incorporating these findings and of the C5a-C5aR+C3a-C3aR loop are depicted schematically in FIG. 1.

As shown in the Examples of the present application, it was found that blocking C5a/C3a-C5aR/C3aR interactions using either C5aR and C3aR antagonists or adding antagonists (e.g., mAbs) to their C5a and C3a ligands reduces T-cell activation in T-cell mediated disorders, such T-cell mediated corneal inflammation (e.g., Herpes stromal keratitis) and reduce, mitigate, and/or inhibit T-cell differentiation and T-cell mediated inflammation. This shows that complement antagonists (e.g., competitive inhibitors, mABs, interfering RNA) used in combination will not only suppress T-cell differentiation in patients but reduce, inhibit, and/or mitigate adverse effects resulting from T-cell mediated inflammation. Based on these discoveries, the present invention provides a method of treating T-cell mediated disorders and particularly to a method of treating in a subject T-cell mediated ophthalmic or ocular disorders, such T-cell mediated corneal inflammation associated with, for example Herpes stromal keratitis.

The method of the present invention can include administering to a T-cell or antigen presenting cell (APC) that expresses at least one of C5aR or C3aR at least one complement antagonist that inhibits or substantially reduces activity of a complement component and substantially reduces or inhibits the activity T-cell differentiation and/or substantially reduces or inhibits T-cell expression and/or generation of inflammatory cytokines, such as IL-2 and IFNγ. By inhibiting or substantially reducing the activity of a complement component, it is meant that the activity of the complement component may be entirely or partly diminished. For example, an inhibition or reduction in the functioning of a C3/C5 convertase may prevent cleavage of C5 and C3 into C5a and C3a, respectively. An inhibition or reduction in the functioning of C5, C3, C5a and/or C3a polypeptides may reduce or eliminate the ability of C5a and C3a to bind C5aR and C3aR, respectively. An inhibition or reduction in Factor B, Factor D, properidin, Bb, Ba and/or any other protein of the complement pathway that is used in the formation of C3 convertase, C5 convertase, C5, C3, C5a and/or C3a may reduce or eliminate the ability of C5a and C3a to be formed and bind to C5aR and C3aR, respectively. Additionally, an inhibition or reduction in the functioning of a C5aR or C3aR may similarly reduce or eliminate the ability of C5a and C3a to bind C5aR and C3aR, respectively.

In an aspect of the invention, the at least one complement antagonist can include an antibody or antibody fragment directed against a complement component that can affect or inhibit the formation of C3a and/or C5a (e.g., anti-Factor B, anti-Factor D, anti-C5, anti-C3, ant-C5 convertase, and anti-C3 convertase) and/or reduce C5a/C3a-C5aR/C3aR interactions (e.g., anti-C5a, anti-C3a, anti-C5aR, and C3aR antibodies). In one example of the present invention, the antibody or antibody fragment can be directed against or specifically bind to an epitope, an antigenic epitope, or an immunogenic epitope of a C5, C3, C3a, C5a, C5aR, C3aR, C5 convertase, and/or C3 convertase. The term “epitope” as used herein can refer to portions of C5, C3, C3a, C5a, C5aR, C3aR, C5 convertase, and/or C3 convertase having antigenic or immunogenic activity. An “immunogenic epitope” as used herein can include a portion of a C5, C3, C3a, C5a, C5aR, C3aR, C5 convertase, and/or C3 convertase that elicits an immune response in a subject, as determined by any method known in the art. The term “antigenic epitope” as used herein can include a portion of a polypeptide to which an antibody can immunospecifically bind as determined by any method well known in the art.

Examples of antibodies directed against C5, C3, C3a, C5a, C5aR, C3aR, C5 convertase, and/or C3 convertase are known in the art. For example, mouse monoclonal antibodies directed against C3aR can include those available from Santa Cruz Biotechnology, Inc. (Santa Cruz, Calif.). Monoclonal anti-human C5aR antibodies can include those available from Research Diagnostics, Inc. (Flanders, N.J.). Monoclonal anti-human/anti-mouse C3a antibodies can include those available from Fitzgerald Industries International, Inc. (Concord, Me.). Monoclonal anti-human/anti-mouse C5a antibodies can include those available from R&D Systems, Inc. (Minneapolis, Minn.).

In another aspect of the invention, the complement antagonist can include purified polypeptide that is a dominant negative or competitive inhibitor of C5, C3, C3a, C5a, C5aR, C3aR, C5 convertase, and/or C3 convertase. As used herein, “dominant negative” or “competitive inhibitor” refers to variant forms of a protein that inhibit the activity of the endogenous, wild type form of the protein (i.e., C5, C3, C3a, C5a, C5aR, C3aR, C5 convertase, and/or C3 convertase). As a result, the dominant negative or competitive inhibitor of a protein promotes the “off” state of protein activity. In the context of the present invention, a dominant negative or competitive inhibitor of C5, C3, C3a, C5a, C5aR, C3aR, C5 convertase, and/or C3 convertase is a C5, C3, C3a, C5a, C5aR, C3aR, C5 convertase, and/or C3 convertase polypeptide, which has been modified (e.g., by mutation of one or more amino acid residues, by posttranscriptional modification, by posttranslational modification) such that the C5, C3, C3a, C5a, C5aR, C3aR, C5 convertase, and/or C3 convertase inhibits the activity of the endogenous C5, C3, C3a, C5a, C5aR, C3aR, C5 convertase, and/or C3 convertase.

In an aspect of the invention, the competitive inhibitor of C5, C3, C3a, C5a, C5aR, C3aR, C5 convertase, and/or C3 convertase can be a purified polypeptide that has an amino acid sequence, which is substantially similar (i.e., at least about 75%, about 80%, about 85%, about 90%, about 95% similar) to the wild type C5, C3, C3a, C5a, C5aR, C3aR, C5 convertase, and/or C3 convertase but with a loss of function. The purified polypeptide, which is a competitive inhibitor of C5, C3, C3a, C5a, C5aR, C3aR, C5 convertase, and/or C3 convertase, can be administered to a T cell or APC expressing C5aR and/or C3aR.

It will be appreciated that antibodies directed to other complement components used in the formation of C5, C3, C5a, C3a, C5 convertase, and/or C3 convertase can be used in accordance with the method of the present invention to reduce and/or inhibit interactions C5a and/or C3a with C5aR and C3aR on the T cells or APCs. The antibodies can include, for example, known Factor B, properdin, and Factor D antibodies that reduce, block, or inhibit the classical and/or alternative pathway of the complement system.

In a further aspect of the present invention, the at least one complement antagonist can include RNA interference (RNAi) polynucleotides to induce knockdown of an mRNA encoding a complement component. For example, an RNAi polynucleotide can comprise an siRNA capable of inducing knockdown of an mRNA encoding a C3, C5, C5aR, or C3aR polypeptide in the T cell.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3 end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Examples of a siRNA molecule directed to an mRNA encoding a C3a, C5a, C5aR, or C3aR polypeptide are known in the art. For instance, human C3a, C3aR, and C5a siRNA is available from Santa Cruz Biotechnology, Inc. (Santa Cruz, Calif.). Additionally, C5aR siRNA is available from Qiagen, Inc. (Valencia, Calif.). siRNAs directed to other complement components, including C3 and C5, are known in the art.

In other embodiments, the RNAi construct can be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Examples RNAi constructs that specifically recognize a particular gene or a particular family of genes, can be selected using methodology outlined in detail above with respect to the selection of antisense oligonucleotide. Similarly, methods of delivery RNAi constructs include the methods for delivery antisense oligonucleotides outlined in detail above.

In some embodiments, a lentiviral vector can be used for the long-term expression of a siRNA, such as a short-hairpin RNA (shRNA), to knockdown expression of C5, C3, C5aR, and/or C3aR in T cells or APCs. Although there have been some safety concerns about the use of lentiviral vectors for gene therapy, self-inactivating lentiviral vectors are considered good candidates for gene therapy as they readily transfect mammalian cells.

It will be appreciated that RNAi constructs directed to other complement components used in the formation of C5, C3, C5a, C3a, C5 convertase, and/or C3 convertase can be used in accordance with the method of the present invention to reduce and/or inhibit interactions C5a and/or C3a with C5aR and C3aR on the T cells or APCs. The RNAi constructs can include, for example, known Factor B, properdin, and Factor D siRNA that reduce expression of Factor B, properdin, and Factor D.

Moreover, it will be appreciated that other antibodies, small molecules, and/or peptides that reduce or inhibit the formation of C5, C3, C5a, C3a, C5 convertase, and/or C3 convertase and/or that reduce or inhibit interactions C5a and/or C3a with C5aR and C3aR on the T-cells can be used as a complement antagonist in accordance with the method of the present invention. These other complement antagonists can be administered to the subject and/or T cells at amount effective to T-cell expression of inflammatory cytokines. Example of such other complement antagonists include C5aR antagonists, such as AcPhe[Orn-Pro-D-cyclohexylalanine-Trp-Arg, prednisolone, and infliximab (Woodruff et al., The Journal of Immunology, 2003, 171: 5514-5520), hexapeptide MeFKPdChaWr (March et al., Mol Pharmacol 65:868-879, 2004), PMX53 and PMX205, and N-[(4-dimethylaminophenyl)methyl]-N-(4-isopropylphenyl)-7-methoxy-1,2,3,4-tetrahydronaphthalen-1-carboxamide hydrochloride (W-54011) (Sumichika et al., J. Biol. Chem., Vol. 277, Issue 51, 49403-49407, Dec. 20, 2002), and a C3aR antagonist, such as SB 290157 (Ratajczak et al., Blood, 15 Mar. 2004, Vol. 103, No. 6, pp. 2071-2078).

The at least one complement antagonist can be administered to the T-cells or APCs, either in vivo or in vitro. The cell can be derived from a human subject, from a known cell line, or from some other suitable source. One example of a cell can include a lymphocyte located in, for example, in tissue of a human subject. The cell may be isolated or, alternatively, associated with any number of identical, similar, or different cell types. Where the cell comprises a lymphocyte, for example, the lymphocyte may be associated with a costimulatory cell, such as an APC. The lymphocyte can be located near or proximate an inflamed tissue in the subject and the complement antagonist can be used to treat T-cell mediated inflammation in the subject.

In one aspect of the invention, the complement antagonist used in methods of the present invention can be administered to the subject to treat corneal inflammation using standard methods including, for example, ophthalmic, topical, parenteral, subcutaneous, intravenous, intraarticular, intrathecal, intramuscular, intraperitoneal, intradermal injections, or by transdermal, buccal, oromucosal, oral routes or via inhalation. The particular approach and dosage used for a particular subject depends on several factors including, for example, the general health, weight, and age of the subject. Based on factors such as these, a medical practitioner can select an appropriate approach to treatment.

Treatment according to the present methods of the invention can be altered, stopped, or re-initiated in a subject depending on the status of corneal inflammation. Treatment can be carried out as intervals determined to be appropriate by those skilled in the art. For example, the administration can be carried out 1, 2, 3, or 4 times a day. In another aspect of the present invention, a complement antagonist can be administered after induction of the inflammatory response has occurred.

The methods of the present invention include administering to the subject a therapeutically effective amount of a complement antagonist. Determination of a therapeutically effective amount is within the capability of those skilled in the art. The exact formulation, route of administration, and dosage can be chosen by the individual physician in view of the subject's condition.

Formulation of pharmaceutical compounds for use in the modes of administration noted above (and others) are described, for example, in Remington's Pharmaceutical Sciences (18^{th} edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999.

In one example, the complement antagonist can be provided in ophthalmic preparation that can be administered to the subject's cornea or eye. The ophthalmic preparation can contain a complement antagonist in a pharmaceutically acceptable solution, suspension or ointment. Some variations in concentration will necessarily occur, depending on the particular complement antagonist employed, the condition of the subject to be treated and the like, and the person responsible for treatment will determine the most suitable concentration for the individual subject. The ophthalmic preparation can be in the form of a sterile aqueous solution containing, if desired, additional ingredients, for example, preservatives, buffers, tonicity agents, antioxidants, stabilizers, nonionic wetting or clarifying agents, and viscosity increasing agents.

Examples of preservatives for use in such a solution include benzalkonium chloride, benzethonium chloride, chlorobutanol, thimerosal and the like. Examples of buffers include boric acid, sodium and potassium bicarbonate, sodium and potassium borates, sodium and potassium carbonate, sodium acetate, and sodium biphosphate, in amounts sufficient to maintain the pH at between about pH 6 and about pH 8, and for example, between about pH 7 and about pH 7.5. Examples of tonicity agents are dextran 40, dextran 70, dextrose, glycerin, potassium chloride, propylene glycol, and sodium chloride.

Examples of antioxidants and stabilizers include sodium bisulfite, sodium metabisulfite, sodium thiosulfite, and thiourea. Examples of wetting and clarifying agents include polysorbate 80, polysorbate 20, poloxamer 282 and tyloxapol. Examples of viscosity-increasing agents include gelatin, glycerin, hydroxyethylcellulose, hydroxmethylpropylcellulose, lanolin, methylcellulose, petrolatum, polyethylene glycol, polyvinyl alcohol, polyvinylpyrrolidone, and carboxymethylcellulose. The ophthalmic preparation will be administered topically to the eye of the subject in need of treatment by conventional methods, for example, in the form of drops or by bathing the eye in the ophthalmic solution.

The complement antagonists can also be formulated for topical administration through the skin. “Topical delivery systems” also include transdermal patches containing the ingredient to be administered. Delivery through the skin can further be achieved by iontophoresis or electrotransport, if desired.

Formulations for topical administration to the skin include, for example, ointments, creams, gels and pastes comprising the complement antagonist in a pharmaceutical acceptable carrier. The formulation of complement antagonists for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

Subjects that are treated according to the methods of the present invention include those who have a T-cell mediated ophthalmic or ocular disorders, such as T-cell mediated corneal inflammation. In addition, subjects who do not have, but are at risk of developing corneal inflammation can be treated according to the methods of the present invention. In the latter group of subjects, the treatment can inhibit or prevent the development of T-cell mediated corneal inflammation in the subject.

In one aspect of the present invention, the T-cell mediated inflammation treated by the methods described herein are related to an ocular disorder, such as ischemic retinopathies in general, anterior ischemic optic neuropathy, all forms of optic neuritis, age-related macular degeneration (AMD), in its dry forms (dry AMD) and wet forms (wet AMD), diabetic retinopathy, diabetic macular edema (DMe.), proliferative diabetic retinopathy (PDR), cystoid macular edema (CMe.), retinal detachment, retinitis pigmentosa (RP), Stargardt's disease, Best's vitelliform retinal degeneration, Leber's congenital amaurosis and other hereditary retinal degenerations, pathologic myopia, retinopathy of prematurity, and Leber's hereditary optic neuropathy, the after effects of corneal transplantation or of refractive corneal surgery, keratoconjunctivitis sicca (KCS) or dry eye, Sjogren's syndrome, conjunctivitis, keratitis, and herpes stromal keratitis. In one specific example, the method can be used to treat T-cell mediated corneal inflammation associated with Herpes stromal keratitis.

In another aspect of the invention, the methods described herein can be used to treat sterile T-cell mediated corneal inflammation in which no living organisms are recovered from either a contact lens or the corneal surface. More specifically, the methods of the present invention can be used to treat T-cell mediated corneal inflammation in a subject associated with contact lens wear. These syndromes can include, but are not limited to Contact Lens Associated Corneal Infiltrates (CLACI), Contact Lens Associated Red Eye (CLARE), Contact Lens Peripheral Ulcer (CPLU). Sterile and infectious infiltrates can usually, but not always, be distinguished by slit lamp examination by those having ordinary skill in the art.

In yet another aspect, the complement antagonists described herein can be administered as part of a combinatorial therapy with additional therapeutic agents. The phrase “combinatorial therapy” or “combination therapy” embraces the administration of a complement antagonist, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

For example, the combinational therapy can include the administration of a complement antagonist with at least one antibacterial, antiviral or antifungal agent to treat corneal inflammation. Examples of anti-bacterials include Gentamycin, fortified with vancomycin for methicillin-resistant Staphylococcus aureus (MRSA) infections, 4^{th} generation fluroquinoline like moxifloxacin or gatifloxacin, cefazolin or vancomycin and fluoroquinolone. In one specific example, the combinational therapy includes a complement antagonist and at least one ophthalmic antibiotic or ophthalmic antiviral. Ophthalmic antibiotics include, for example, chloramphenicol sodium succinate ophthalmic (chloramphenical); CORTISPORIN (neomycin and polymyxin 13 sulfates and hydrocortisone acetate cream); ILOTYCIN (erythromycin ophthalmic ointment); NEODECADRON (neomycin sulfate-dexamethasone sodium phosphate); POLYTRIM (trimethoprim and polythyxin β sulfate opthalmic solution); TERRA-CORTRIL (oxytetracycline HCL and hydrocortisone acetate); TERRAMYCIN (oxytetratcycline); and TOBRADEX (tobramycin and dexamethosone ophthalmic suspension and ointment).

Ophthalmic antivirals include, for example, VIRA-A ophthalmic ointment, (vidarabine). Opthalmic quinalones include, for example, CHIBROXIN (norfloxacin ophthalmic solution); CILOXAN ophthalmic solution, (Ciprofloxacin HCL); and Ocuflox ophthalmic solution (ofloxacin). Opthalmic sulfonamides include, for example, BLEPHAMIDE ophthalmic ointment (sulfacetamide sodium and prednisolone acetate); and BLEPHAMIDE ophthalmic suspension (sulfacetamide sodium and prednisolone acetate). Antifungals include, for example, natamycin and amphotericin-B.

The present invention further relates to a method of treating a T-cell mediated inflammatory response in a subject's cornea. The method includes administering to the subject a therapeutically effective amount of a complement antagonist. In another aspect of the present invention, the treatment of the T-cell mediated inflammatory response can include the inhibition of T-cell inflammatory cytokine generation.

The present invention also relates to a contact lens for treating T-cell mediated corneal inflammation in a subject. The contact lens includes a contact lens substrate and a coating provided on at least a portion of the substrate. The coating can include an amount of complement antagonist effective to treat corneal inflammation in a subject upon administration of the contact lens to the subject.

Coatings including complement antagonists can be applied to a number of contact lens substrate materials known in the art. Virtually any substrate known in the art that can be fashioned into a contact lens can be used in the present invention provided it is optically transparent.

In an aspect of the invention, the substrate can include optically transparent materials that allow oxygen to reach the cornea in an amount, which is sufficient for long-term corneal health. Examples of substrates include polymers made from hydrophobic materials, such as silicone copolymers, interpolymers, oligomers, and macromers. Illustrative polysilicones are polydimethyl siloxane, polydimethyl-co-vinylmethylsiloxane. Other silicones include silicone rubbers described in U.S. Pat. No. 3,228,741 to Becker; blends such as those described in U.S. Pat. No. 3,341,490 to Burdick et al., and silicone compositions such as described in U.S. Pat. No. 3,518,324 to Polmanteer. Substrates described in U.S. Pat. Nos. 4,136,250; 5,387,623; 5,760,100; 5,789,461; 5,776,999; 5,849,811; 5,314,960 and 5,244,981 can also be used in the invention. Cross-linked polymers of propoxylate of methyl glucose and propylene oxide and HEMA-based hydrogels can also be used as substrates of the contact lens.

Silicone compositions that can be used in forming the contact lens of this invention are the cross-linked polysiloxanes obtained by cross-linking siloxane prepolymers by means of hydrosilylation, co-condensation and by free radical mechanisms such those described by Chen in U.S. Pat. No. 4,143,949, which is incorporated herein by reference. Additional silicone-based substrates are cross-linked polymers of α,ω-bisamionpropyl polydimethylsiloxane, and gylycidyl methacrylate, cross-linked polymers. Silicone compositions also contemplated by the present invention are made from combining a methacrylate with one or more silicone monomers in the presence of a group transfer polymerization (GTP) catalyst to form a macromer that is subsequently polymerized with other monomers to give the final substrate. Initiators, reaction conditions, monomers, and catalysts that can be used to make group transfer (GTP) polymers are described in “Group Transfer Polymerization” by O. W. Webster, in Encyclopedia of Polymer Science and Engineering Ed. (John Wiley & Sons) p. 580, 1987. Substrates described in U.S. Pat. No. 6,951,894 are also suitable for use in the present invention.

The coating can be prepared and applied as an aqueous solution, suspension, or colloid and then applied to the contact lens substrate according to any process that can provide the coating in contact with the substrate. For example, processes for applying the coating to the substrate include immersion, spraying, brushing, and spin coating. Once the lens substrate is coated, it may be subjected to any number of additional steps that are conducted in the manufacture of contact lenses. These can include, for example, swelling and washing steps, the addition of additives such as surfactants, extraction steps and the like.

The coating including the complement antagonist can adhere to the contact lens by, for example, chemical bonding, such as covalent or ionic bonding, or physical bonding. In some aspects, the coating can remain affixed to the lens substrate throughout its useful life (e.g., storage time plus the time in which it will be in contact with a user's eye).

The contact lens can also include more than one layer of coating. This can be desirable where the coating layer will provide the requisite surface properties (e.g. treatment of corneal inflammation) but is not particularly compatible with the lens substrate itself. For example, a tie-layer or coupling agent can be used to adhere the coating to the substrate. Selections of compatible lens substrate complement antagonist coating, and tie-layer (if necessary) materials is well within the knowledge of one skilled in the art.

In aspect of the invention, the contact lens is non-toxic to the subject's cornea and other tissue while providing for the treatment of conical inflammation in the subject.

The present invention also relates to an ophthalmic solution for treating T-cell mediated corneal inflammation in a subject. The solution can be aqueous and include a complement antagonist as described above. Examples of solutions useful that can be used in the treatment of corneal inflammation include solutions that are contacted with eye lids and/or eyes, such as multipurpose lens solutions, opthalmalic rinse solutions, surgical scrubs for eye use, eye drops, eye wash solutions, contact lens solutions, topical over the counter ocular and periocular solutions (i.e. artificial tears), ocular and periocular cleaning solutions, eye irrigating solutions, and /or antibacterial solutions for surgical scrubs or topical application.

In some aspects, a complement antagonist may be added to a commercially available contact lens solution or a multipurpose lens solution to treat conical inflammation. In other aspects, a complement antagonist may be added to an aqueous solution prepared for use as a contact lens or multipurpose lens solution that is not commercially available to treat corneal inflammation.

In some aspect where the ophthalmic solution includes a cleaning solution, the cleaning solution can include cleaning agents to effectively clean a lens of film deposits and surface debris. Examples of cleaning agents that can be used include, poloxamers and tetronic surfactants comprising poly(oxythylene) hydrophilic units. In all embodiments, the cleaning agents are nontoxic, and do not distort the vision of the subject being treated for corneal inflammation.

In other aspects, complement antagonists may be added to tonicity agents and buffers that are found in conventional ophthalmic solutions. Examples of tonicifiers include dextrose, potassium chloride and/or sodium chloride. Examples of buffers include boric acid, sodium borate, sodium or potassium citrate, sodium bicarbonate, sodium phosphate, and potassium phosphate.

Additionally, antibacterial agents found in conventional ophthalmic solutions, such as multipurpose lens solutions, may be added. Antibacterial agents for use in the solution include, for example, polyaminopropyl biguanide, alexidine hydrochloride, polyquatemium-1, polyquatemium 42, myristamidopropyl dimethylamine, or other agents known to those skilled in the art.

In some aspects, the solution may further include a comfort or moisturizing agent to provide hydration and lubrication of a subject's contact lens. Such agents include, for example, polyquatemium 10, poloxamer, propylene glycol, hydroxypropylmethylcellulose (HPMC), or other agents known to those skilled in the art.

Since, in some aspects, the solution is intended to be administered topically to the eyelids and/or eye, it is contemplated that the solution be free of pathogenic organisms and/or sterile. A benefit of a sterile solution is that it reduces the possibility of introducing contaminants into a subject's eyelids and/or eye. Sterility or adequate antimicrobial preservation may be provided as part of the present solutions of the present invention. In some aspects, the solutions are produced under sterile conditions.

In addition to or in place of sterilization, aqueous solutions of the complement antagonist may contain a physiologically acceptable preservative to minimize the possibility of microbial contamination. A physiologically acceptable preservative may be used in the solutions of the present invention to increase the stability of the solutions. Preservatives include, for example, polyaminopropyl biguanide, polyhexamethylene biguanide (PHMB), polyquatemium-1, myristamidopropyl, and sorbic acid.

The following examples are for the purpose of illustration only and are not intended to limit the scope of the claims, which are appended hereto.

### EXAMPLE 1

We initiated studies to determine whether C5a and/or another activation fragment i.e., C3a, generated from complement endogenously produced by cognate APC-T cell partners, participate(s) in T cell differentiation into IFNγ⁺ effector cells. C5a and C3a are ˜10 kDa anaphylatoxins able to ligate the C5a receptor (C5aR) and the C3a receptor (C3aR) that are G protein-coupled receptors (GPCRs) generally expressed on APCs and reported under some conditions to be detectable on T cells (Soruri et al., 2003). We performed studies with primary APCs and T cells by using two independent approaches, genetic deficiency and pharmacological blockade, to assess whether and, if so, how the local complement production relates to physiological T cell responses. After finding that these GPCR engagements indeed are integrally involved in the T cell activation process physiologically, we investigated the mechanism and unexpectedly found that their signaling not only functions integrally in costimulation but also operates constitutively in naive T cells to sustain their viability.

### Results

### Cognate APC-T Cell Partners Locally Generate C5a and C3a and Upregulate C5aR and C3aR

We tested whether APCs and T cells additionally synthesize C5 as well as C5aR and C3aR that could result in local C5a-C5aR and/or C3a-C3aR engagements.

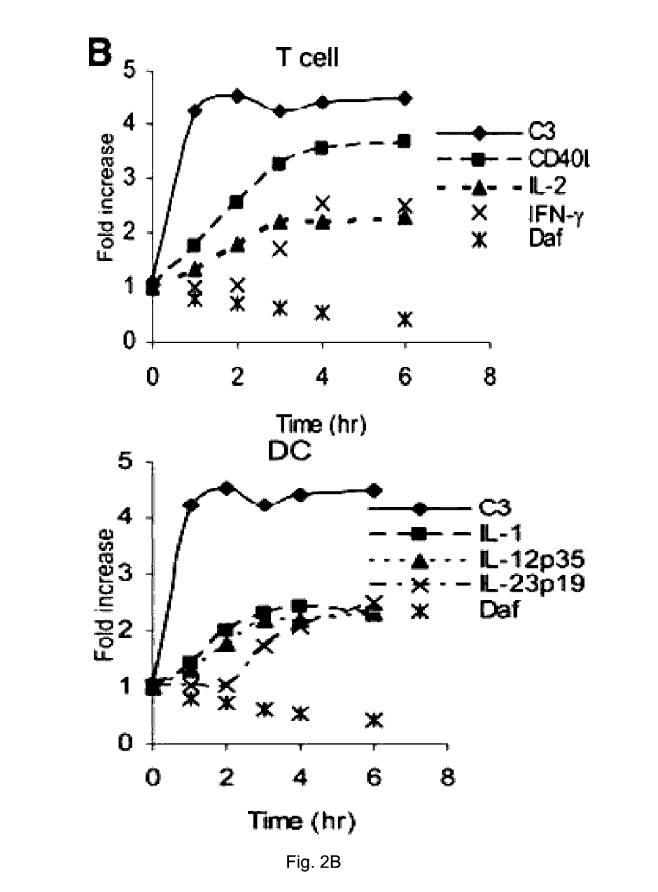

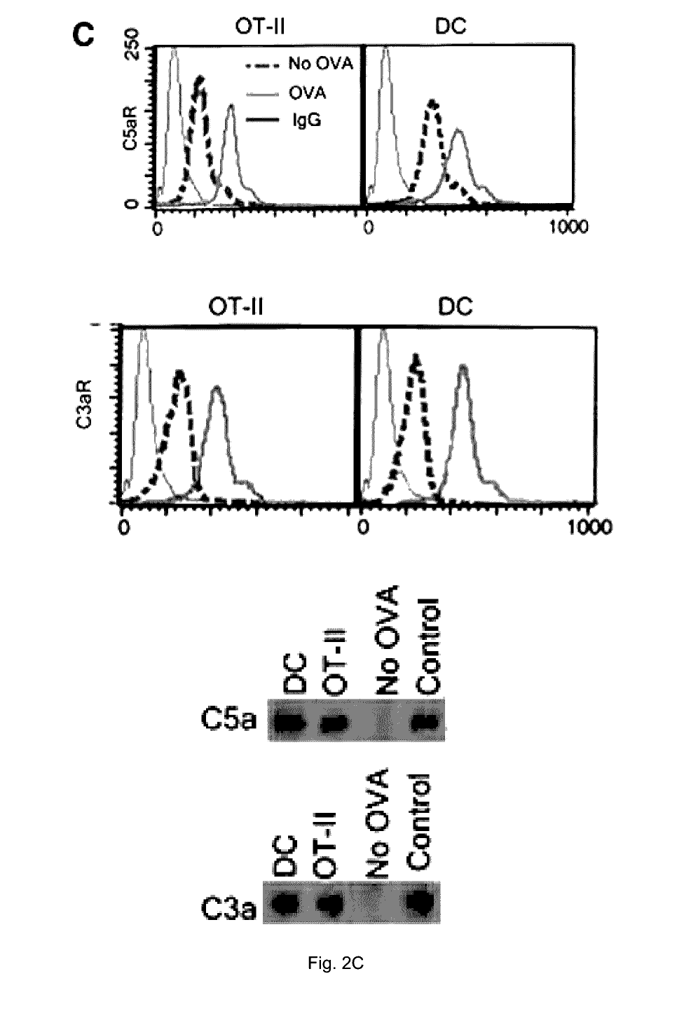

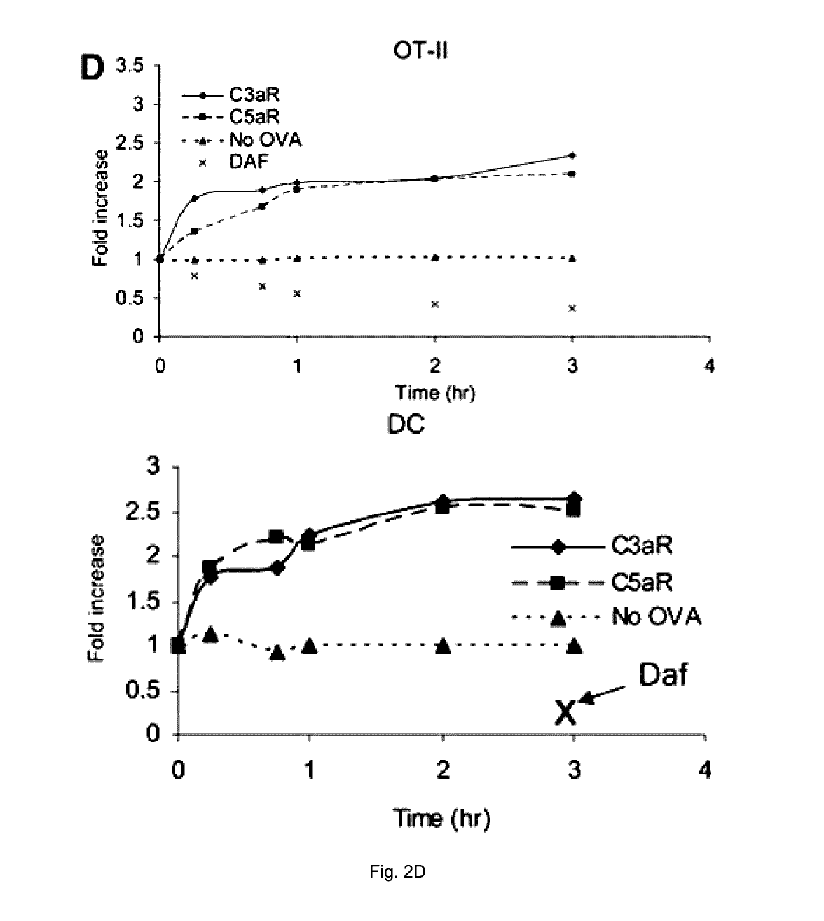

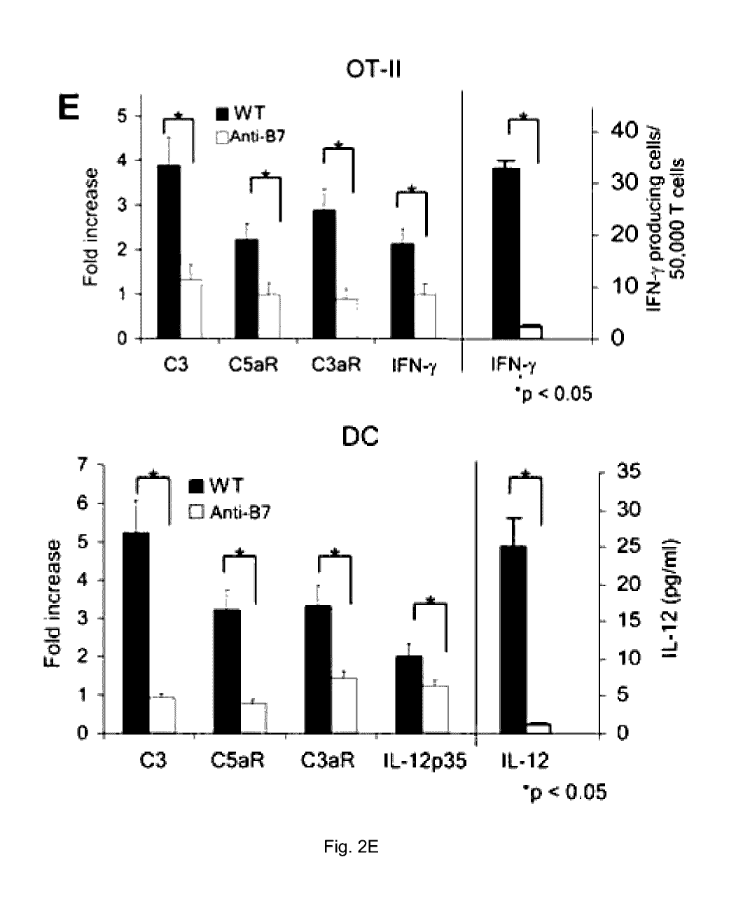

Initially, we incubated OT-II TCR transgenic T cells with OVA₃₂₃₋₃₃₉ peptide plus bone-marrow-derived dendritic cells (DCs) as APCs. At various time points, we flow separated the APCs and T cells, isolated their RNA, and assayed complement-gene-expression patterns in the two partners by qPCR. These assays showed that in addition to upregulating C3, fB, and fD, both the APCs and T cells indeed upregulated mRNAs for C5 as well as C5aR and C3aR (FIG. 2A). Notably, although both partners produced complement, on the basis of a quantitative analysis of per-cell copy number with an internal standard (data not shown), C3 mRNA was produced in 1000-fold excess by the APCs compared to the T cells both at rest and after activation. The analyses also showed that during cognate interactions, T cells as well as the APCs concurrently downregulated Dafl mRNA (FIGS. 2A and 1B), thereby further lowering restraint on local C3, fB, fD, and C5 activation.

Kinetic analyses (FIG. 2B) revealed that the complement upregulation in T cells preceded the well-established, activation-induced upregulation of CD40L mRNA expression, and that both preceded IL-2 mRNA expression. In the DCs, C3 mRNA upregulation occurred much earlier than upregulation of IL-1, IL-12p35, and IL-23p19 mRNAs known to influence T cell differentiation. As expected, the upregulation of IL-12p35 mRNA by the DCs (2-fold at 2 hr) preceded the upregulation of IFNg mRNA in the OT-II cells (2-fold>3 hr).

To determine whether the changes in mRNA translated into differences in protein production, we performed flow-cytometric analyses (FIGS. 2C and 2D). These assays confirmed upregulated expression of C5aR and C3aR levels on both the T cells and APCs. The upregulated surface C5aR and C3aR on both partners persisted in the presence but not absence of OVA peptide (FIG. 2D), documenting antigen dependence Immunoblottings performed on the serum-free culture supernatants showed the ˜10 kB C5a and C3a ligands for C5aR and C3aR (FIG. 2C, right), indicating that the locally produced components underwent spontaneous alternative-pathway activation. The generation of C5a and C3a (which signal at 10⁻¹³ M) and augmentation of C5aR+C3aR on the cell surfaces continued over the ensuing 3 hr and thereafter in the APC-peptide-T cell mixture (FIG. 2D). Concurrently, surface Daf protein expression progressively declined on the T cells as well as on the APCs and was well below baseline at 3 hr. Thus, interacting APC and T cell partners both generate C5a and C3a and upregulate C5aR and C3aR.

### APC-T Cell Complement Component and Receptor Inductions Are Dependent on CD28-B7 and CD40-40L Couplings

To address mechanisms underlying the observed APC-T cell complement component and receptor upregulations, we next tested the impact of costimulation on these processes. Upon addition of blocking B71/2 mAbs, T cell differentiation and cytokine production was diminished (FIG. 2E), APC IL-12 upregulation was prevented, C3, C3aR, and C5aR gene expression on both partners was abrogated, C5aR and C3aR surface upregulation did not occur, and substantially lower amounts C3a and C5a were detected in culture supernatants (not shown). Similarly, after the addition of the blocking anti-CD40L mAb MR-1 but not a control IgG, IFNγ production as well as complement-component and receptor-gene and protein upregulation by the OT-II T cells was prevented. Notably, the abrogation of complement upregulation by the loss of either B7-CD28 or CD40-CD154 interactions was 70%, but if the two costimulatory pairs were blocked simultaneously, the complement-gene expression was suppressed below control values. Similar results were obtained with an independent Marilyn TCR transgenic T cell system specific for HYDby plus I-A^{b} (not shown).

Thus, the data show that cognate T cell-APC interactions along with costimulatory signals (delivered through CD80- and CD86-CD28 and CD40-40L) induce the sustained local production of C5a and C3a, downregulation of cell-surface expression of DAF, and persistent upregulation C5aR and C3aR. All of these events occur prior to T cell proliferation and cytokine secretion.

### Disabling C5aR+C3aR Signaling Prevents T Cell Responses In Vitro

In view of the findings that APC and T cell partners sustain local generation of C5a+C3a together with augmenting their C5aR+C3aR surface expression during cognate interactions, we tested whether signaling through C5aR and/or C3aR by the locally generated C5a and C3a ligands is involved in activation of T cells by APCs.

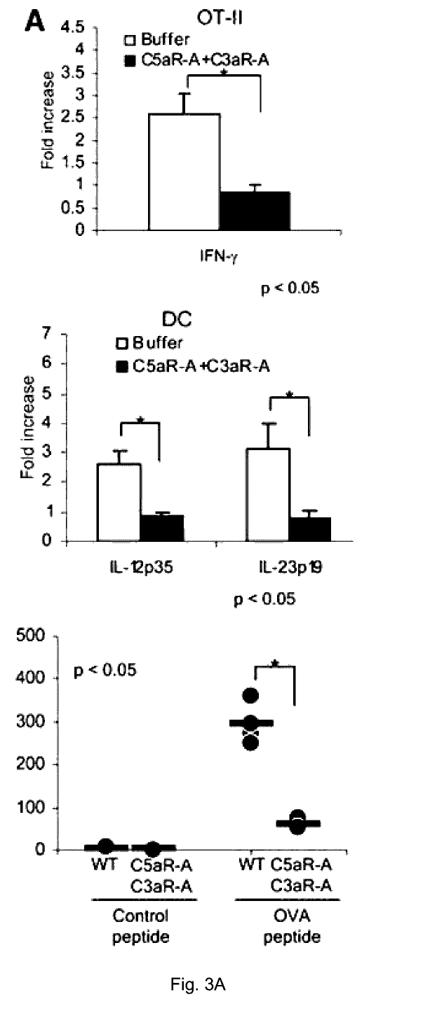

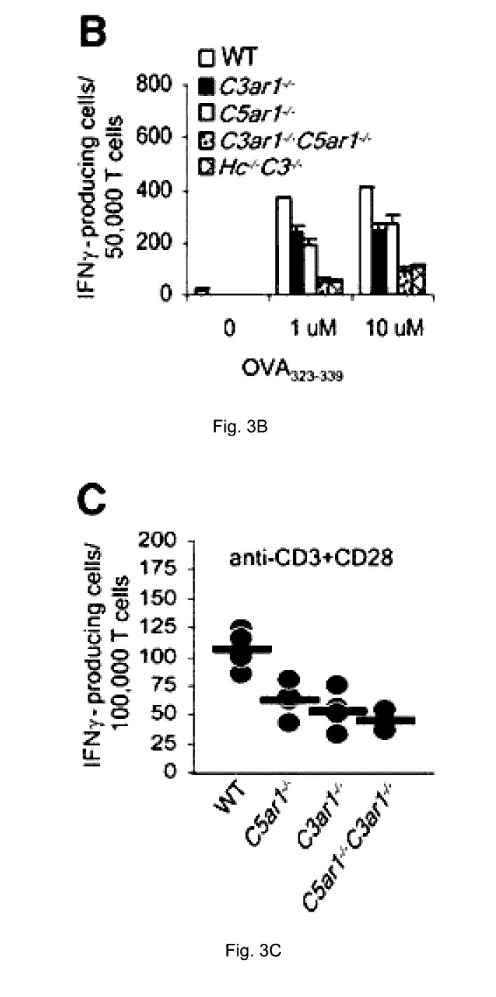

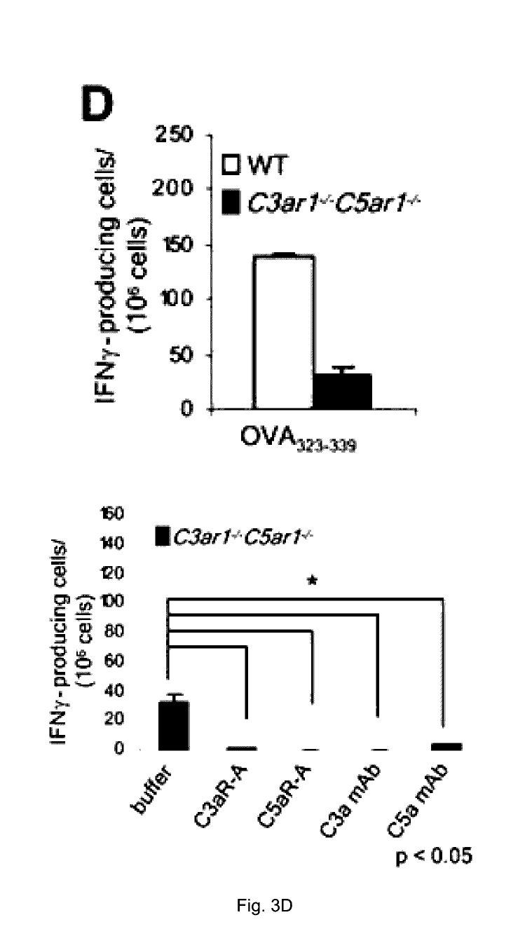

To do this, we performed studies with OT-II cells, OVA₃₂₃₋₃₃₉ peptide, and WT DCs in which we blocked C5a-C5aR+C3a-C3aR engagements on the C5ar1^{+/+}C3ar1^{+/+} OT-II T cells and APCs by the addition of a C5aR antagonist (C5aR-A), a cyclic peptide C5a analog that binds to C5aR and prevents C5aR signaling, and a C3aR antagonist (C3aR-A) that, like the C5aR-A, binds to but does not signal through C3aR. Both antagonists block their target receptors with high specificity. The two antagonists added together prevented upregulation of IFNγ mRNA in the responding T cells (FIG. 3A, left), as well as production of IL-12p35 and IL-23p19 mRNA in the DCs (FIG. 3A, middle). A similar result was obtained with anti-CD3+anti-CD28 stimulation of WT T cells (FIG. 3A, right).

To assess the contribution of C5aR and C3aR function on APCs in mediating T cell reactivity, we mixed WT OT-II T cells with DCs from WT, C5ar1^{-/-}, C3ar1^{-/-}, and C3ar1^{-/-}C5ar1^{-/-} mice. The absence of either receptor from the APC diminished T cell IFNγ production by 30%-50% (FIG. 3B). The impact of the absence of both APC C3aR and C5aR was greater than the absence of either receptor alone in that T cell IFNγ production was inhibited by greater than 75% (FIG. 3B). A comparable result was obtained if APCs from mice lacking C5 and C3 (Hc^{-/-}C3^{-/-}) were used (FIG. 3B). To address the impact of C5aR and C3aR on T cells, we used anti-CD3+anti-CD28 stimulation (FIG. 2C). Whereas this stimulation induced IFNγ production by WT T cells, stimulation of C5ar1^{-/-}C3ar1^{-/-} T cells yielded a markedly reduced response. Similarly, when we employed anti-C5a and anti-C3a mAbs together with C5ar1^{-/-}C3ar1^{-/-} DCs in the DC-OVA₃₂₃₋₃₃₉-OT-II system to block OT-II C3aR and C5aR signaling, it had the same effect (FIG. 3D).

### C3aR and C5aR Control T Cell Immunity In Vivo

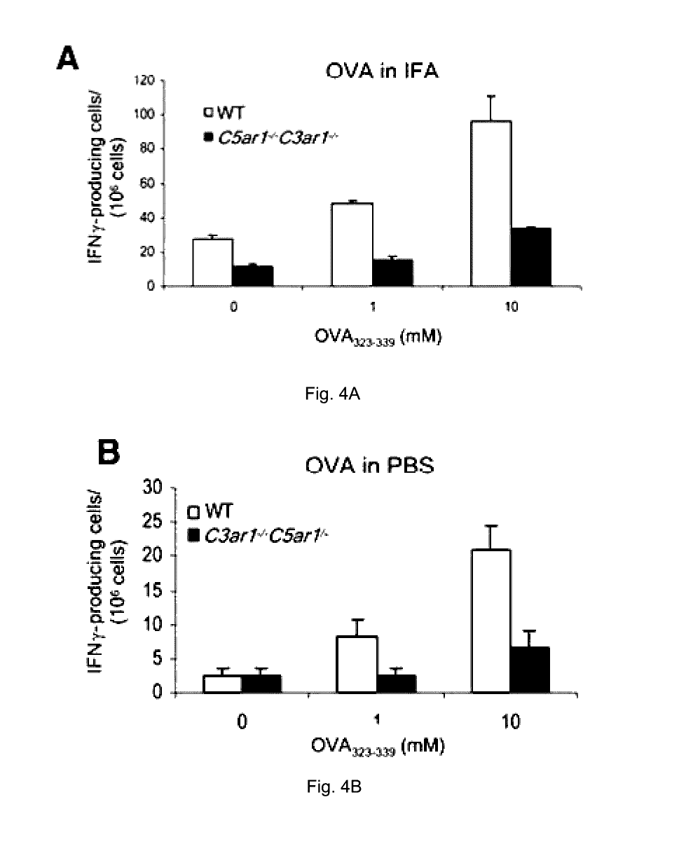

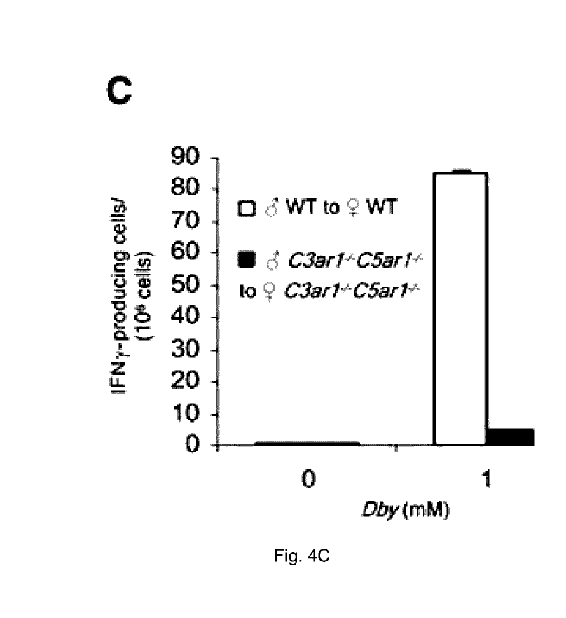

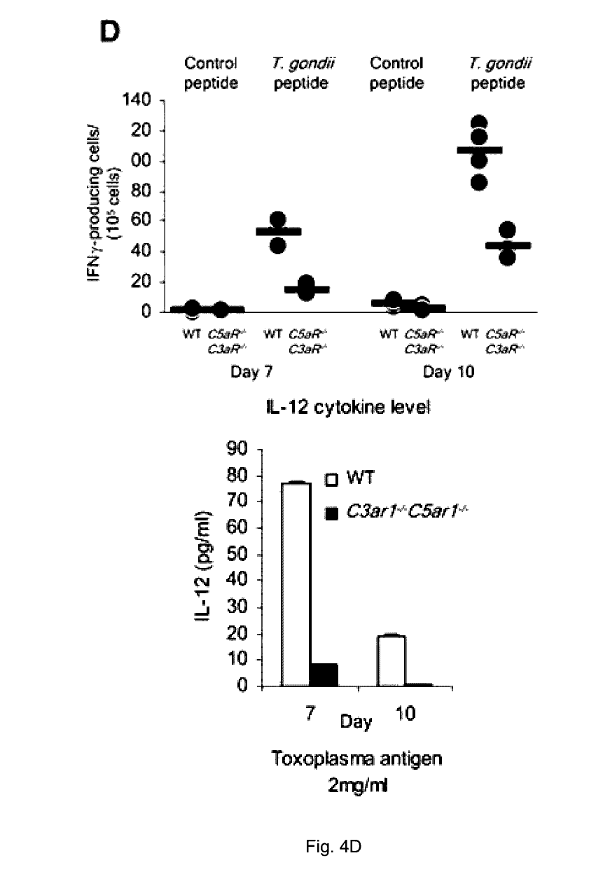

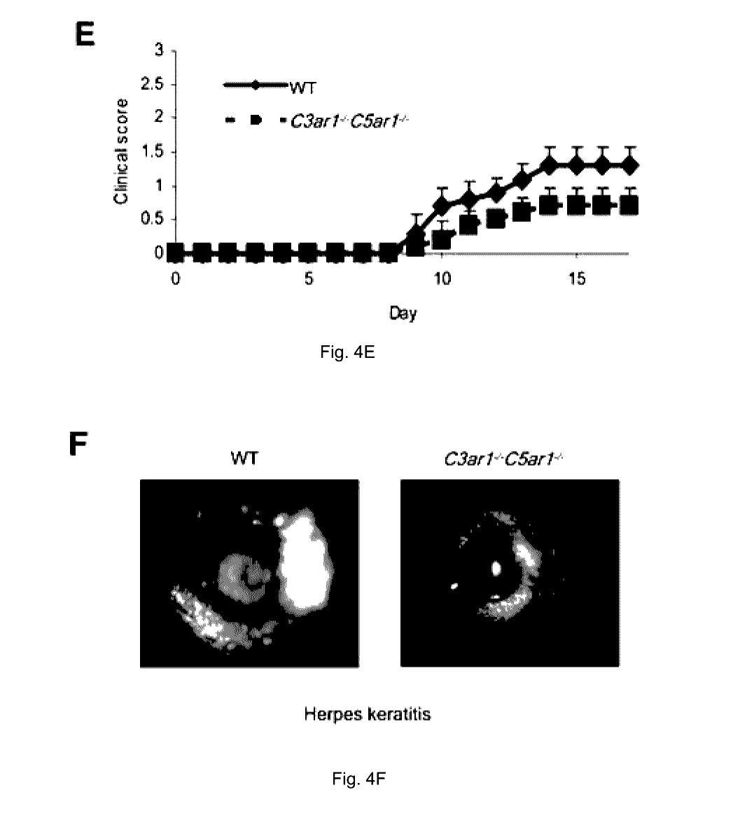

To test the impact of APC-T cell C5a-C5aR+C3a-C3aR signaling in vivo, we performed multiple complementary experiments. After immunization of WT or C5ar1^{-/-}C3ar1^{-/-} mice with ovalbumin protein mixed in incomplete Freund's adjuvant (IFA), we found that recall responses by ELISPOT assay on day 10 were reduced by 50% in C5ar1^{-/-}C3ar1^{-/-} mice compared to WTs (FIG. 4A). To assess immunity in the absence of adjuvant, we immunized mice with ovalbumin in PBS. As anticipated, the responses (FIG. 4B) were lower, but again the frequency of specific IFNγ-producing T cells was reduced by 80% in C5ar1^{-/-}C3ar1^{-/-} mice. With another approach, we injected male cells into syngeneic H-2b WT or H-2b C5ar1^{-/-}C3ar1^{-/-} mice. Whereas HYDby responses to the HYDby (I-A^{b}-restricted determinant) 2 weeks later were detected after the WT male transfers, none were detected with the male C5ar1^{-/-}C3ar1^{-/-} transfers (FIG. 4C). In another model system, 40% of CFSE-labeled OT-II T cells underwent at least one round of proliferation after adoptive transfer into WT mice primed 24 hr previously with OVA₃₂₃₋₃₃₉ in CFA, but <5% underwent at least one round of proliferation in identically primed C5ar1^{-/-}C3ar1^{-/-} mice (p<0.05 versus WT, n=2 per group, not shown). Similar results were obtained in an allo model in which CFSE-labeled Balb/c T cells were adoptively transferred to WT and Hc^{-/-}C3^{-/-} mice (20% versus <2% undergoing one or more rounds of proliferation, n=2, not shown). In another adoptive-transfer model, IFN-γ producing cells generated by C5ar1^{-/-}C3ar1^{-/-} C57BL/6 female Mar T cells into C5ar1^{-/-} male recipients were reduced by 33% of those generated by WT female Mar T cells injected into male C57BL/6 recipients (not shown).

To evaluate the impact of C3aR and C5aR in clinically relevant disease models, we infected WT and C5ar1^{-/-}C3ar1^{-/-} mice with T. gondii (ME49 strain), a system in which disease protection is IL-12 and T cell dependent. The C5ar1^{-/-}C3ar1^{-/-} mice survived for only 12 days, whereas all simultaneously infected WT mice appeared clinically healthy on day 12 (n=5 per group) and survived for >50 days (not shown). Spleen cells from the WT animals sacrificed on both day 7 and 10 post-infection prior to appearing ill on day 12 responded strongly to T. gondii antigens as assessed by IL-12 levels in culture supernatants and a high frequency of antigen-specific IFNγ producers (FIG. 4D, left). In contrast, at both time points, spleen cells from the infected C5ar1^{-/-}C3ar1^{-/-} mice contained 70% fewer IFNγ-producing cells (p<0.05) together with a complete lack of IL-12 secretion (FIG. 4D, right). In the T cell dependent MOG35-55-induced experimental autoimmune encephalomyelitis (EAE) model of multiple sclerosis, WTs showed characteristic weakness (clinical score >1.5), whereas C5ar1^{-/-}C3ar1^{-/-} mice showed less weakness (clinical score 0.5) that persisted over time (n=4 per group, p<0.05). In a herpes keratitis model, in which inflammation is due to host T cell responses against virally infected corneal cells rather than the virus itself, corneas of WT mice completely opacified at day 14, whereas corneas of C5ar1^{-/-}C3ar1^{-/-} mice showed little or no change (n=5, p<0.005; FIG. 4F). Thus in all systems, both in vitro and in vivo, T cell immunity in the absence of C5aR or C3aR on APCs or T cells was markedly impaired.

### C5a-C5aR+C3a-C3aR Engagements Function as Autocrine Regulators

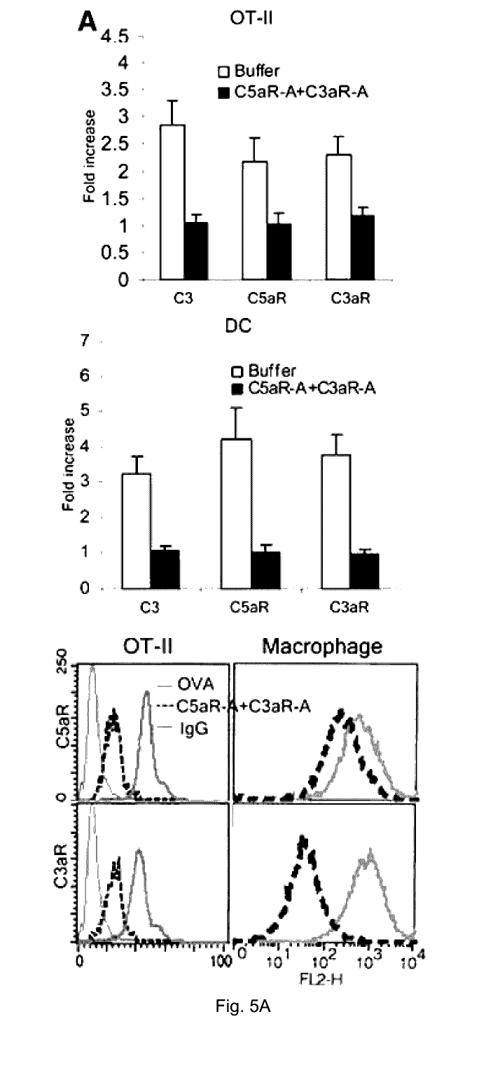

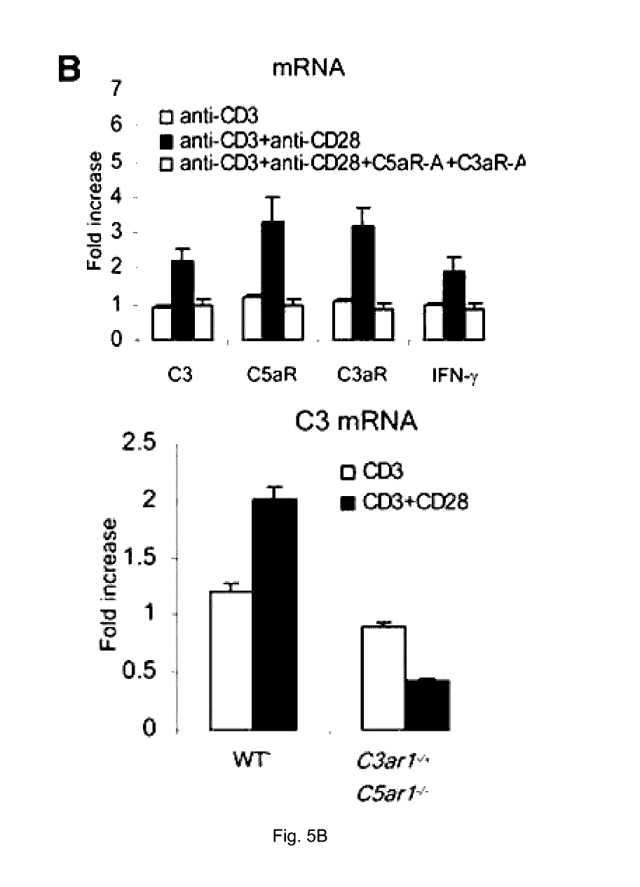

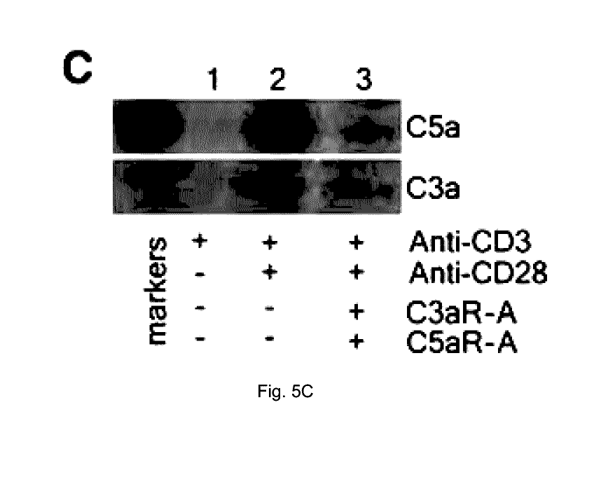

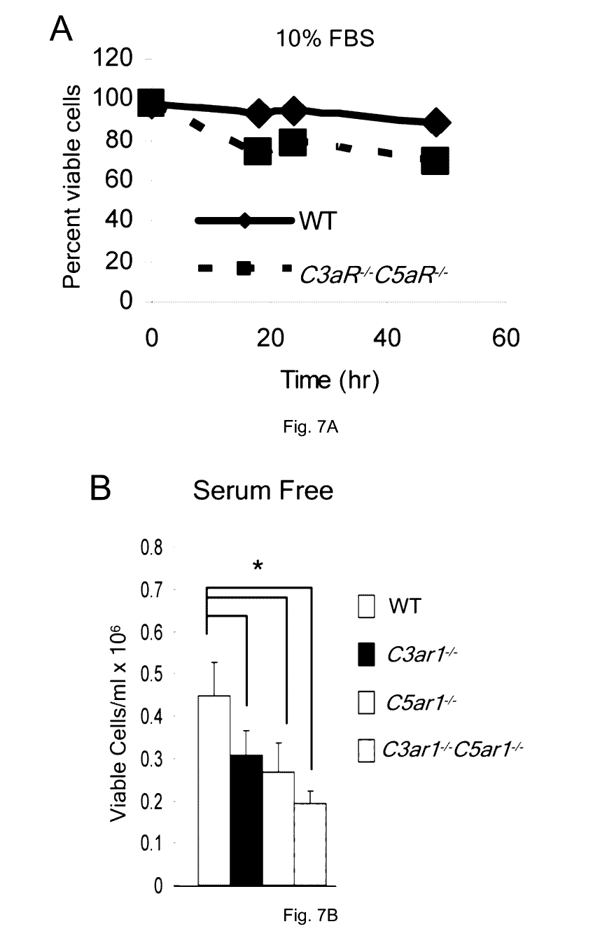

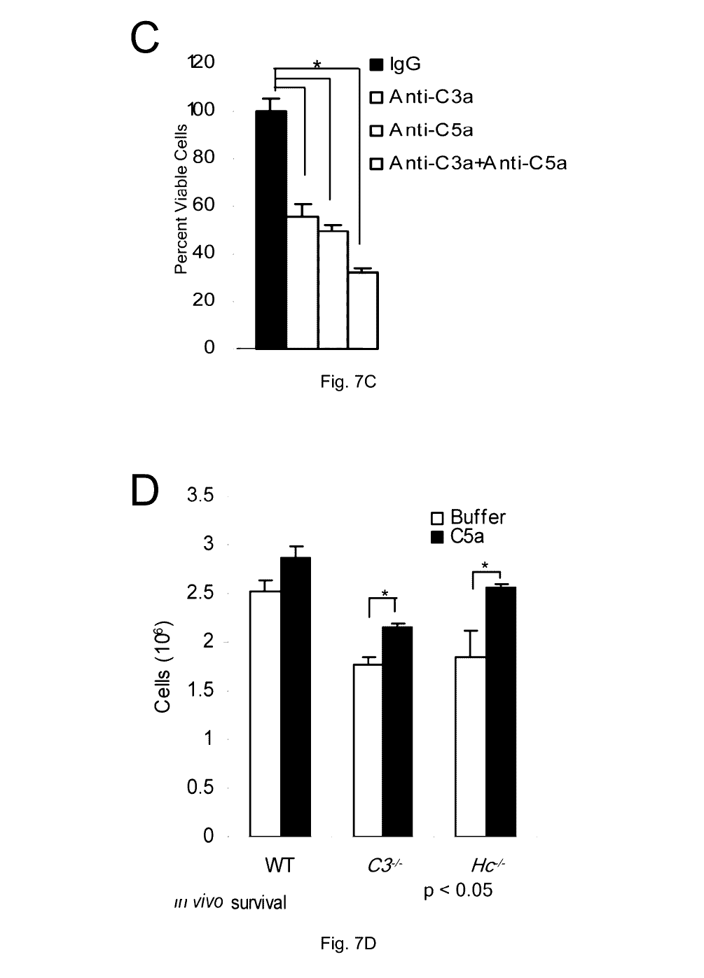

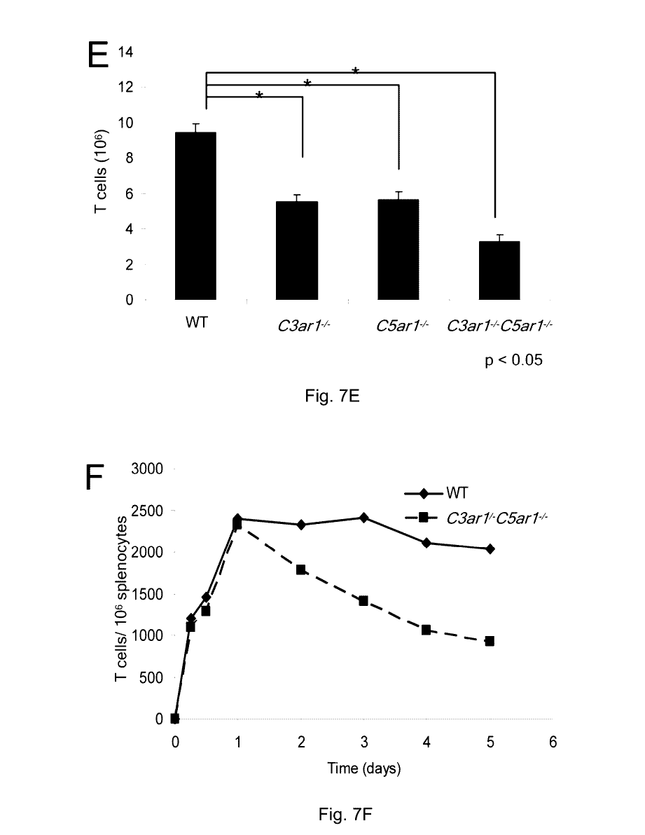

The finding that complement upregulation was maintained for prolonged periods upon initial antigen stimulation (FIG. 2) suggested the possibility of an autocrine feedback loop in which cognate interactions induce complement production and initiate activation and locally produced C3a and C5a maintain upregulated production of these components in both partners by virtue of signaling through their C5aR+C3aR. Consistent with this, blockade of C3aR+C5aR signaling with the specific antagonists not only prevented OT-II T cell proliferation and cytokine secretion (FIG. 3) but also prevented the induced upregulation of complement components and receptors in both partners (FIG. 5A). Moreover, anti-CD3+anti-CD28 stimulation of OT-II cells induced complement-component-gene upregulation in WT T cells (FIG. 5B), but the same treatment in the presence of the C3aR-A and C5aR-A, or upon stimulation of C5ar1^{-/-}C3ar1^{-/-} T cells had no effect (FIG. 5B) Immunoblots of culture supernatants of anti-CD3+anti-CD28-stimulated OT-II cells showed both C5a and C3a (FIG. 5C), but >90% reduced C5a+C3a generation after 2 hr of incubation with C5aR-A and C3aR-A. Overexposures of unstimulated T cells in medium alone showed low-level C5a and C3a production (lane 1), an intriguing result that will be addressed below (see FIG. 7). Analogously, APCs deficient in both C3aR and C5aR did not upregulate gene expression for complement components during cognate interactions with TCR tg T cells. The C5ar1^{-/-}C3ar1^{-/-} DCs showed no differences from WTs in cell numbers or viability (not shown).

To further assess autocrine effects of C5a-C5aR+C3a-C3aR, we evaluated T cell responses in a system in which either the T cell or the APC, or both, were C5aR+C3aR disabled. As shown in FIG. 3B, the generation of IFNγ-producing cells was reduced to 20% when C5ar1^{-/-}C3ar1^{-/-} DCs were used, whereas it was reduced to <5% if the C5aR-A and C3aR-A or anti-05a/anti-C3a mAbs were added to block C5aR+C3aR signals in the OT-II cells. Similarly, the WT Balb/c T cell proliferation in response to B6 C3^{-/-} APCs was reduced to 50% of B6 WT APCs, and C3^{-/-} Balb/c T cell proliferation was reduced to 50% in response to C3^{-/-} than WT APCs, indicating that C3 production by the T cell as well as the APC participates in proliferation. The findings, taken together with those above, indicate that C3, fB, fD, and C5 produced by either partner during cognate interactions can activate (i.e., generate C3a and C5a) and in turn function in both a paracrine and autocrine manner to perpetuate local complement production and drive T cell responses. In the absence of C5aR+C3aR from either partner (in this serum-free in vitro system), no C5a or C3a is produced, preventing T cell proliferation and differentiation.

### Disabling C5aR+C3aR Signaling Reduces Costimulatory-Molecule Expression

To clarify how disabled C5aR+C3aR signaling diminishes T cell responses, we examined costimulatory-molecule expression patterns by flow cytometry. These analyses showed decreased surface expression of B7, CD40, and class II MHC (not shown) on peritoneal macrophages from C5ar1^{-/-} and C3ar1^{-/-} mice and essentially no staining on C5ar1^{-/-}C3ar1^{-/-} DCs. Parallel analyses of C5aR-A- and C3aR-A-treated WT macrophages showed similar reductions in costimulatory-molecule expression. Consistent with this, in the absence of C3aR and C5aR on the APC, the cognate T cell APC interaction failed to induce upregulation of IL-1, IL-23, and IL-12.

We also examined CD28 and CD40L expression on WT and C5ar1^{-/-}C3ar1^{-/-} T cells by flow cytometry. T cell surface expression of CD28 was reduced by >90% on resting C5ar1^{-/-}C3ar1^{-/-} than on WT T cells, and CD40L levels were similarly reduced after anti-CD3+anti-CD28 stimulation. Parallel analyses of C5aR-A- and C3aR-A-treated WT T cells showed similar reductions.

### Costimulatory-Molecule Expression Is Dependent Upon C5aR+C3aR Signaling

Costimulatory-molecule interactions are essential for optimal APC induction of T cell responses. To establish whether locally produced C5a+C3a and upregulated expression of C5aR+C3aR (FIG. 2) by APC-T cell partners and costimulatory-molecule expression are mechanistically linked, we tested whether abrogation of T cell responsiveness in the absence of costimulatory-molecule signaling can be reversed by C3a+C5a addition. Consistent with this, diminished proliferation of Mar CD4+ T cells (Dby plus I-A^{b}) in response to Cd80^{-/-}Cd86^{-/-} DCs was reversed upon addition of C5a, added C5a reconstituted diminished IFNγ production by OT-II cells stimulated with CD40^{-/-} APCs, and addition of anti-CD3 plus exogenous C5a led to a response equal that of anti-CD3+anti-CD28. Consistent with the above implicated autocrine loop (FIG. 5), the exogenous C5a also induced upregulation of local complement synthesis comparably to that measured after anti-CD3+anti-CD28 stimulation and greater than that detected after anti-CD3 stimulation alone. Whereas C3ar1^{-/-} cells responded to exogenous C5a, C5ar1^{-/-} T cells did not respond, demonstrating that the effects are mediated through this receptor (as opposed to the alternative C5a receptor, C5L2.

Strong support for a link between costimulation, T cell reactivity, local complement production, and C5aR+C3aR GPCR signaling derives from the observation that diminished B7 and CD28 expression on C3ar1^{-/-} (but not C5ar1^{-/-}) DCs and T cells, respectively, was restored to WT values after either C5a or C3a addition. Likewise, C5a addition to C3^{-/-}Hc^{-/-} T cells restored CD28 expression whereas anti-C5a+anti-C3a addition to WT T cells decreased CD28 expression. To directly confirm that both APC and T cell costimulatory-molecule expression is controlled by C5aR and C3aR GPCR signaling, we added human C5a to C5aR expressing THP1 cells transfected with a luciferase reporter driven by the promoter of B7.1. The added C5a induced a luciferase signal indicative of C5aR dependent regulation. Overall, these data show that CD80, CD86 and CD40 costimulation yield local complement production and subsequent generation of C5a+C3a. The resultant C5a and C3a fragments signal through their GCPRs to upregulate costimulatory-molecule expression and directly drive T cell proliferation and differentiation.

C5a-C5aR+C3a-C3aR Signaling Is Linked to T Cell Activation through P13-Kg-Induced Phosphorylation of AKT

Studies with Jurkat cells have shown that B7-CD28 ligation signals in T cells through phosphorylation of Y¹⁷⁰ residue in the YMNM motif in CD28's cytoplasmic tail, permitting SH2-dependent binding and activation of PI-3 kinase p85αp110α (PI-3Kα). The activated PI-3Kα increases the amount of internal leaflet-associated phosphatidylinositol 3,4,5 trisphosphate [PtdIns (3,4,5)P₃], causing the recruitment of PDK1, PDK2, and AKT via their pleckstrin homology (PH) domains [which enable them to bind PtdIns (3,4,5)P₃]. This juxtaposition on PtdIns (3,4,5)P₃ allows PDK1+PDK2 to produce dually Thr₃₀₈Ser₄₇₃ phosphorylated AKT that is centrally implicated in CD28 costimulatory signaling.

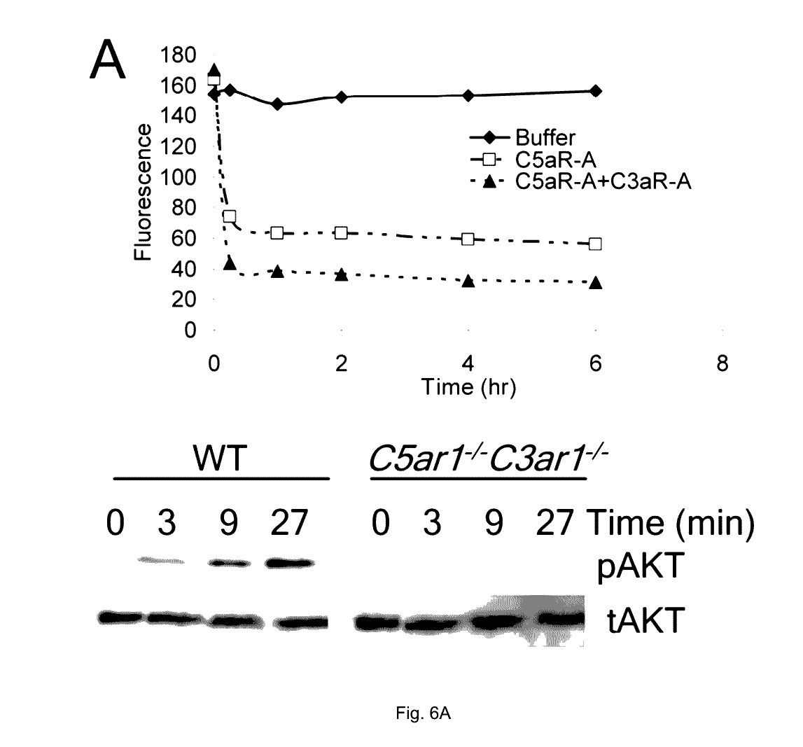

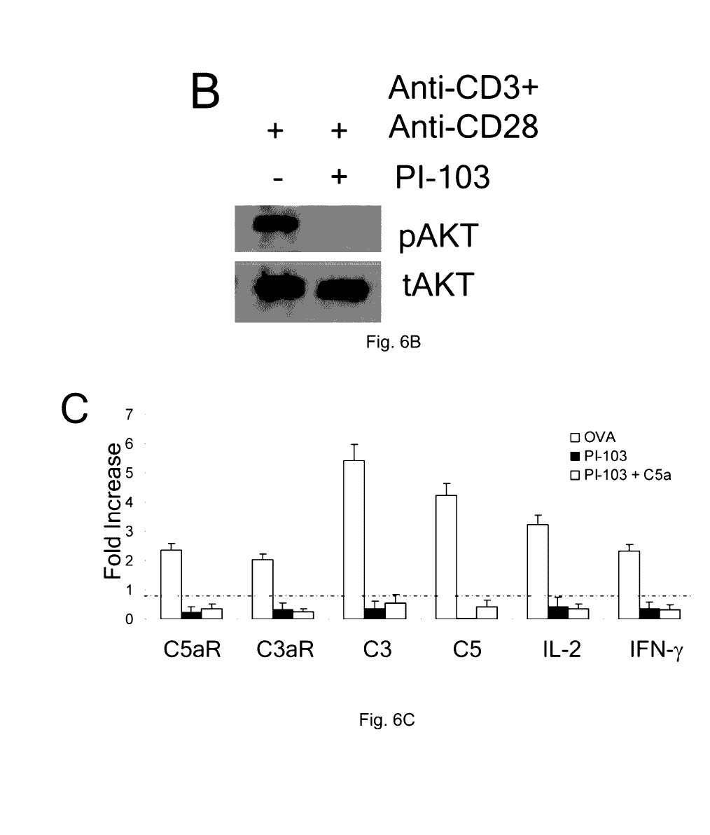

To test whether abrogated CD28 costimulation in the absence or blockade of C5aR+C3aR relates to a requirement for these GCPR signals for optimal AKT phosphorylation, we stimulated WT T cells with anti-CD3+anti-CD28±C5aR-A and C3aR-A and assessed AKT phosphorylation by immunoblotting and Luminex assay (FIG. 6A). Notably, whereas addition of the C5aR-A diminished AKT phosphorylation, both antagonists together virtually abolished it. Moreover, significantly less phosphorylated AKT was detectable upon anti-CD3+anti-CD28 stimulation of C5ar1^{-/-}C3ar1^{-/-} T cells at all time points tested (FIG. 6B).

AKT is one product resulting from the activity of the p110 catalytic subunit of PI-3 kinase p101γp110γ (PI-3Kγ), and PI-3Kγ has been tied to GCPR signal transduction in neutrophils and macrophages. To test whether this signaling pathway is operational in T cells, we incubated WT T cells with anti-CD3+anti-CD28 plus a specific inhibitor (PI-103) of PI-3Kγ. Strikingly, addition of the inhibitor (1) abrogated anti-CD3+anti-CD28-induced phosphorylation of AKT (FIG. 6B), (2) prevented the upregulation of complement gene expression, and (3) eliminated upregulation of IL-2 and IFNγ mRNA expression (FIG. 6C). Consistent with PI-3Kγ activation being mediated through C5aR ligation, these suppressive effects could not be overcome by added C5a (FIG. 6C). Thus, C5aR- and C3aR-induced PI-3Kγ activation is necessary for AKT phosphorylation and resultant T cell activation.

### C5aR+C3aR Signaling Is Essential for Sustaining Naive T Cell Viability

We detected low levels of C5a+C3a in culture supernatants of T cells in the absence of stimulation (FIG. 7B), a result raising the possibility that C5a+C3a is generated constitutively and is important for T cell function. Moreover, we observed reduced S⁴⁷³ AKT phosphorylation at early time points after preincubation of mouse T cells with the C5aR-A and C3aR-A (FIG. 6A), further implicating C5a-C5aR+C3a-C3aR interactions as operating tonically in naive T cells. The two results would be consistent with preexisting C5a+C3a feeding back on C5aR+C3aR to trigger ongoing basal GPCR activation. In support of this, C5aR and C3aR could be detected on unstimulated T cells with an ultrabright chromophore, WT but not C5ar1^{-/-}C3ar1^{-/-} T cells produced C5a and C3a at rest (FIG. 7A), and as noted in FIG. 6A, basal phospho S⁴⁷³ AKT was readily detectable in WT T cells but was markedly reduced in C5ar1^{-/-}C3ar1^{-/-} T cells (FIG. 6B). Because of the known association of loss of phospho S⁴⁷³ AKT with induction of programmed cell death (PCD), this connection of disabled C5aR+C3aR GPCR signaling in naive unstimulated cells with reduced phospho S⁴⁷³ AKT suggested the intriguing possibility that in naive T cells, constitutive C5a-C5aR+C3a-C3aR interactions play a role in maintaining viability.

To test this possibility, we compared the survival of WT and C5ar1^{-/-}C3ar1^{-/-} T cells in vitro after 48 hr of incubation in 10% heat-inactivated FCS. These analyses showed that in contrast to about 5% loss of WT T cells, 20%-30% loss of C5ar1^{-/-}C3ar1^{-/-} T cells occurred (FIG. 7A). To eliminate effects of exogenous complement, growth factors, and other agents in serum, we repeated the survival studies with serum-free medium. Fewer surviving C5ar1^{-/-}C3ar1^{-/-} T cells were detected at 6 hr, confirming that disrupted autocrine signaling through both the C5aR and C3aR contributed to the decline (FIG. 7B). To confirm that these effects were not reflective of other processes, we incubated naive WT T cells in serum-free medium containing anti-C3a and/or anti-C5a mAbs against the C5aR+C3aR ligands rather than blocking the receptors themselves (FIG. 7C). This caused a diminution in cell numbers similar to that observed with C5ar1^{-/-}C3ar1^{-/-} cells. As yet another test, we added C5a to C5-deficient or C3^{-/-} T cells. This augmented 6 hr viability of both knockouts by 25%, close to the viability of WT cells (FIG. 7D). Thus, tonic C5a-C5aR+C3a-C3aR signal transduction is necessary for maintaining T cells in a viable state.

### The In Vivo Half-Lives of Naive T Cells Are Shorter in the Absence of C5aR+C3aR Signaling

To document that the relationship between C5a-C5aR+C3a-C3aR interactions and viability is relevant physiologically, we compared the number of CD3⁺ T cells in spleens of nave C5ar1^{-/-}, C3ar1^{-/-}, and C5ar1^{-/-}C3ar1^{-/-} mice to those in spleens of WT mice. The cell counts revealed 2-, 2-, and 3-fold fewer C5ar1^{-/-}, C3ar1^{-/-}, and C5ar1^{-/-}C3ar1^{-/-} T cells, respectively, than WT CD3⁺ T cells per spleen (FIG. 7E). Because total cell number at steady state is a reflection of production and destruction, we performed the more direct test of in vivo cell survival. We coadoptively transferred equal numbers of nave CellTracker Red CMTPX-labeled C5ar1^{-/-}C3ar1^{-/-} and CFSE-labeled WT T cells together into SCID mice and determined the number of viable cells in spleens after the transfer (FIG. 7F). Similar numbers of each T cell population were detectable 8-24 hr, indicating that the cells equally migrated to spleens after the injection. However, significantly fewer C5ar1^{-/-}C3ar1^{-/-} T cells were detectable in the recipient spleens on days 2-5, consistent with the conclusion that viability is reduced. Control studies in which the cell-membrane labels were switched yielded the same results (data not shown). These studies thus documents that tonic C5a-C5aR and C3a-C3aR signal transduction functions in vivo to maintain the viability of naive T cells.

### EXPERIMENTAL PROCEDURES

### Reagents and Antibodies

Murine C5a was from Cell Sciences (Canton, Me.). Mouse C3a and C5a mAbs were from R&D Systems (Minneapolis, Minn.). Mouse IL-4, GM-CSF, and M-CSF were from Peprotech (Rockyhill, N.J.). Antibodies against mouse B7-1 and B7-2 were from BD PharMingen (San Diego, Calif.). Anti-CD40L mAb was from Bio Express (West Lebanon, N.H.). Anti-C5aR and anti-C3aR were purchased from Santa Cruz Biotech (Santa Cruz, Calif.). The PI-3K inhibitors were provided by Dr. Kevin Shokat. Peptides were synthesized by Research Genetics as described.

### Animals

C57BL/6, OT-II (specific for OVA₃₂₃₋₃₃₉ plus I-A^{b}), Cd80^{-/-}Cd86, C3^{-/-}, C5-deficient, and CD40^{-/-} mice were from Jackson labs (Bar Harbor, Me.). C3^{-/-} mice and C3ar1^{-/-} and C5ar1^{-/-} were gifts of Dr. Michael Carroll and Dr. Craig Gerard (Harvard Medical School and Children's Hospital, Boston, Mass.). Marilyn (MAR) transgenic was a gift of Polly Matzinger, Ghost Lab, NIH. We generated Hc^{-/-}C3^{-/-} mice by crossing C5-deficient B10.2 mice with C57BL/6 congenic C3^{-/-} mice. C5^{+/+}C3^{+/+} littermates used as controls displayed comparable results to the studies with C57BL/6 mice as controls. All studies were approved by the Case Western Reserve University Institutional Animal Care and Use Center (IACUC).

RNA Purification, cDNA Synthesis, and qPCR

Cells were purified for 5 min at 20° C. with Trizol (Invitrogen, Carlsbad, Calif.) according to the manufacturer's instructions. When C3aR and C5aR mRNAs were analyzed, preparations were treated with DNase I (standard protocol) for removal of genomic DNA. We synthesized cDNAs by incubating 20 μl of mRNAs in Sprint PowerScript Single Shots (Clontech, Mountain View, Calif.). A total of 10 μl of diluted cDNA were mixed with 2 μl of primer and 10 μl SYBR green master mix (Applied Biosystems, Foster City, Calif.) and assayed in triplicate on an ABI prism 7000 cycler. In all assays, fold increases are relative to each basal level and standardized to Actin.

### Murine DCs and T Cells

Bone-marrow cells were grown in RPMI 1640/10% FBS containing 10 μg/ml IL-4+10 μg/ml GM-CSF. Fresh media with the same cytokines was added on day 3, 10 μg/ml IL-4 and 5 μg/ml GM-CSF were added on day 5, and cells were used on day 6. T cells harvested from spleens were purified with T cell enrichment columns (R&D Systems).

### Immunizations, ELISPOT Assays and CFSE Proliferation, and Flow Cytometry

Mice were immunized s.c. with OVA₃₂₃₋₃₃₉ peptide as described (Heeger et al., 2005). ELSPOT and proliferation assays were performed as described (Heeger et al., 2005). All antibodies were purchased from BD PharMingen (San Diego, Calif.), and stained cells were analyzed on a Becton Dickinson LSR II.

### Anti-CD3 and Anti-CD28 Stimulations

Cells were stimulated for 1 hr with 1 mg/ml anti-CD3 and/or anti-CD28 (BD Biosciences) in serum-free RPMI 1640 for qPCR analyses and for 72 hr for IFNγ ELISPOT assays.

### Immunoprecipitations

Cells were washed twice with PBS and extracted on ice for 10 min with 1% NP-40, 150 mM NaCl, 1 mM PMSF, 0.4 mM EDTA, and a protease-inhibitor cocktail (Complete Mini, Roche, Mannheim, Germany). After centrifugation of extracts for 10 mM at 13,000×g, supernatants were incubated for 1 hr at 4° C. with appropriate antibody, after which Sepharose A beads were added and the mixture incubated overnight at 4° C. Centrifuged pellets were washed 5×, SDS sample buffer was added, and boiled samples were loaded onto SDS-PAGE gels.

### Immunoblotting

All blots were performed by standard procedure as described (Lin et al., 2001) with HRP-conjugated secondary antibody and an ECL enhancer (GE Healthcare, Buckinghamshire, UK).

Quantitation of Murine C3 mRNA

A cDNA library was made from a C57BL/6 liver. The C3 standard was amplified with the qPCR primer for C3 via conventional PCR and diluted to 10⁶ copies/mL. A standard curve was created with 10-fold dilutions of the C3 standard and assayed by qPCR as above alongside with the cDNA libraries from total RNA isolated from the T cells and DCs. A standard curve was constructed from the CT values of the C3 standard, and the copies/mL of the samples were determined. We used the amount of total RNA from each sample to determine the amount of copies/cell.

### Luminex Assay

Cells were stimulated for increasing times with 1 mg/ml anti-CD3+anti-CD28 mAbs. After stimulation, cells were assayed for pAKT and tAKT with Upstate's Beadlyte assay according to the manufacturer's instructions (Upstate, NY). In brief, cells were placed on ice immediately after incubation, centrifuged at 4° C., lysed in the buffer provided by the company, incubated with the capture beads and then the detection beads, washed, and assayed on the Bioplex 2200 (Biorad, Hercules, Calif.).

### Luciferase Activity Assay

The base pairs +72 to −991 of the human B7.1 promoter were inserted into a luciferase reporter vector (GL4) then transfected into THP-1 cells by electroporation (×10⁶ cells in 200 μl OptiMEM at 250 V and 950 μF). Cells were incubated overnight in RPMI 1640 and 10% FBS, after incubation at 37° C. for 2 hr with 300 nM C5a in serum-free RPMI 1640; luciferase activity was measured with an Lmax Luminometer (Molecular Devices).

### In Vitro Cell Viability

Mouse T cells purified by EasySep magnetic bead cocktails (StemCell Technologies, British Columbia) were cultured in 96-well plates in serum-free HL-1 media containing L-glutamine and penicillin+streptomycin for the indicated times or were cultured in complete RPMI 1640 (5% FBS, L-glutamine, penn/strep). In some experiments, live and dead cells were counted with trypan blue (Invitrogen, Carlsbad, Calif.). In others, cells were stained with Cy5-anti-CD4/CD8, FITC-anti-CD44, and propidium iodide, mixed with Flow-Check Fluorsperes (Beckman Coulter, Miami, Fla.), and analyzed on a LSR II flow cytometer. Samples were normalized to 1000 Flow Check bead events.

### In Vivo Cell Viability

CD4⁺ T cells from WT mice were labeled with CFSE (Invitrogen), and C5ar1^{-/-}C3ar1^{-/-} mice were labeled CellTracker Red CMTPX (Invitrogen); afterward, 2×10⁶ of each type was injected via tail vein into SCID mice. At various time points, two mice from each group were sacrificed, and total spleen cells were assayed for percentage of labeled cells by flow cytometry.

### Toxoplasmosis Infections

WT or C5ar1^{-/-}C3ar1^{-/-} mice were infected i.p. with 20 cysts of T. gondii (ME49 strain; n=5). The C5ar1^{-/-}C3ar1^{-/-} mice and a parallel set of WT animals (n=5 per group) were killed on day 10-12 (just before death), and spleen cells were isolated, stimulated with toxoplasma gondii antigen for 48 hr, and tested for IL-12 production by ELISA or IFNγ by ELISPOT.

### EXAMPLE 2

We examined the role of the complement system in host immune and inflammatory responses that occur in the cornea in herpes simplex stromal keratitis (HSK).

### Materials and Methods

### Mice & Infection

Male and Female wild-type (WT), C3−/−, C3aR−/−, C5aR−/−, and DAF−/− Balb/c at 6-8 weeks of age were used in these experiments. The cornea of the right eye of the mice were scarified with 27-gauge needle in a crisscross pattern. 1×106 PFU of the KOS strain of HSV type 1 applied topically. The KOS strain has been shown in prior studies to induce HSK within 1-week post infection. The mice were sacrificed day 14 after infection and sent for routine histology.

### HSK Scoring

Mice were examined at days 1, 3, 9, and 14 post-infection and scored as follows:
0: normal cornea
1+: opacity, edema, and neovascularization in less than 25% of the cornea
2+: opacity, edema, and neovascularization in 25% to 50% of the cornea;
3+: opacity, edema, and neovascularization in 50 to 75% of the cornea;
4+: opacity, edema, and neovascularization in 75% to 100% of the cornea

### Results

As shown in FIGS. 8-10 mice that are deficient of C3, C5aR, and C3aR develop less severe disease than WT. Mice deficient of Daf show an increased rate of development of stromal keratitis. Depleting locally produced complement factors C3a and C5a prevents activation of T cells, precluding development of HSK. Depleting Daf results in increased C3a and C5a levels, increasing T cell response and HSK disease.

From the above description of the invention, those skilled in the art will perceive improvements, changes and modifications Such improvements, changes and modifications are within the skill of the art and are intended to be covered by the appended claims. All publications, patents, and patent applications cited in the present application are herein incorporated by reference in their entirety.

1-33. (canceled)

34. A method of treating T-cell mediated corneal inflammation in a subject in need thereof, the method comprising: topically administering to the cornea of the subject at least one complement antagonist that substantially reduces the interaction of C3a with a C3a receptor (C3aR) of a T cell and C5a with a C5a receptor (C5aR) of the T-cell.

35. The method of claim 34, wherein the at least one complement antagonist includes an antibody directed against C5aR and an antibody directed against C3aR.

36. The method of claim 34, the complement antagonist being topically administered to the cornea in an ophthalmic preparation.

37. The method of claim 36, the ophthalmic preparation comprising a contact lens, ophthalmic rinse solution, multipurpose lens solution, eye wash solution, ocular solution, or eye irrigating solution.

38. The method of claim 34, the complement antagonist being applied to a contact lens substrate that is topically administered to the cornea of the subject.

39. The method of claim 34, wherein the T cell mediated inflammation is associated with contact lens wear.

40. A method of treating corneal inflammation associated with contact lens wear in a subject in need thereof, the method comprising: topically administering to the cornea of the subject at least one complement antagonist that substantially reduces the interaction of C3a with a C3a receptor (C3aR) of a T cell and C5a with a C5a receptor (C5aR) of the T-cell.

41. The method of claim 40, wherein the at least one complement antagonist includes an antibody directed against C5aR and an antibody directed against C3aR.

42. The method of claim 40, the complement antagonist being topically administered to the cornea in an ophthalmic preparation.

43. The method of claim 42, the ophthalmic preparation comprising a contact lens, ophthalmic rinse solution, multipurpose lens solution, eye wash solution, ocular solution, or eye irrigating solution.

44. The method of claim 40, the complement antagonist being applied to a contact lens substrate that is topically administered to the cornea of the subject.
